(19) United States
(12) Patent Application Publication
(10) Pub. No.: US 2021/0017492 A1
(43) Pub. Date: Jan. 21, 2021

(54) USE OF RETINOIC ACID IN T-CELL MANUFACTURING

(71) Applicant: Immatics US, Inc., Houston, TX (US)

(72) Inventor: Thorsten DEMBERG, Houston, TX (US)

(21) Appl. No.: 17/062,170

(22) Filed: Oct. 2, 2020

Related U.S. Application Data
(63) Continuation of application No. 16/839,881, filed on Apr. 3, 2020.
(60) Provisional application No. 62/829,485, filed on Apr. 4, 2019.

(51) Int. Cl. C12N 5/0783; C07K 16/28; A61K 35/17

(52) U.S. Cl. CPC C12N 5/0636; C07K 16/2809; C12N 2500/30; C07K 16/2818; A61K 35/17

(57) ABSTRACT

The present disclosure generally relates to methods of improving T cell homing to organs or tissues by utilizing compounds and compositions, for example, retinoic acid receptor agonists. In an aspect, the disclosure provides for organ-homing engineered T cells for treating diseases, associated compositions, and methods for preparing thereof.

### CROSS-REFERENCE TO RELATED APPLICATIONS

This patent application is a Continuation of U.S. application Ser. No. 16/839,881, filed Apr. 3, 2020, which claims priority to U.S. Provisional Patent Application No. 62/829,485, filed Apr. 4, 2019, the disclosure of which is incorporated herein in its entirety.

### REFERENCE TO SEQUENCE LISTING SUBMITTED AS A COMPLIANT ASCII TEXT FILE (.TXT)

Pursuant to the EFS-Web legal framework and 37 C.F.R. § 1.821-825 (see M.P.E.P. § 2442.03(a)), a Sequence Listing in the form of an ASCII-compliant text file (entitled “Sequence_Listing_3000011-009002_ST25.txt” created on Sep. 29, 2020, and 24,495 bytes in size) is submitted concurrently with the instant application, and the entire contents of the Sequence Listing are incorporated herein by reference.

### FIELD OF THE INVENTION

The present disclosure generally relates to methods of improving T cell homing to organs or tissues by retinoic acid receptor agonists. In an aspect, the disclosure provides for organ-homing engineered T cells for treating diseases, compositions comprising the same, and methods for preparing thereof.

### BACKGROUND OF THE INVENTION

While cancer immune therapy has revolutionized the treatment of metastatic disease across a wide range of cancer diagnoses, a limiting factor remains directing or homing T cells to the appropriate tissue or organ with a high level of specificity. The solution to this technical problem is provided by embodiments described herein.

### SUMMARY OF VARIOUS EMBODIMENTS OF THE INVENTION

The disclosure provides for methods of utilizing a compound or composition described herein, such as retinoic acid, in T cell manufacturing. In another aspect, a compound or composition described herein, such as retinoic acid, facilitates the directed homing of T cells to organs or tissues.

In another aspect, the disclosure relates to an improved method of T cell homing in organs, including contacting T cells or a population of T cells with an agonist or antagonist of a retinoic acid receptor in an amount sufficient to modulate homing of T cells to an organ. T cells obtained using retinoic acid have an increased homing to organs, e.g., lung, heart, liver, pancreas, and/or intestine, as compared to T cells obtained without using retinoic acid.

Accordingly, the methods of the present invention provide inter alia (i) T-cells, in particular genetically modified T-cells, wherein the expression of homing markers on the cell surface of said T cells facilitates the directed homing of T cells to organs or tissues, (ii) a T-cell population or T-cells comprising a high proportion of T cells that may be classified as memory T-like cell, which are known to exhibit several disadvantages described herein, improving their use in therapy.

In an aspect, the disclosure relates to methods for genetically modifying cells, including, thawing frozen peripheral blood mononuclear cells (PBMC), resting the thawed PBMC, activating the cultured PBMC with at least one antibody, transducing, transfecting, or electroporating the activated T cells, expanding the transduced, transfected, or electroporated PBMC, and obtaining the expanded T cell, in which at least one of the activating, the transducing, transfecting, or electroporating, and the expanding are performed in the presence of retinoic acid. T cells obtained using this method involving retinoic acid have an increased homing to organs, e.g., lung, heart, liver, pancreas, and/or intestine, as compared to T cells obtained without using retinoic acid.

In another aspect, the disclosure relates to methods for genetically modifying cells, including, thawing frozen peripheral blood mononuclear cells (PBMC), resting the thawed PBMC for about 1 to about 12 hours, activating the cultured PBMC with an anti-CD3 antibody and an anti-CD28 antibody, transducing the activated T cells with a viral vector, expanding the transduced PBMC, and obtaining the expanded T cell, in which at least one of the activating, the transducing, and the expanding are performed in the presence of retinoic acid. The viral vector may be a retroviral vector, adenoviral vector, adeno-associated viral vector, or lentiviral vector.

In another aspect, the disclosure relates to methods of preparing a T cell population, including obtaining fresh peripheral blood mononuclear cells (PBMC), e.g., the fresh PBMC is not cryopreserved, activating the T cell in the fresh PBMC with an anti-CD3 antibody and an anti-CD28 antibody, transducing the activated T cell with a viral vector, expanding the transduced T cell, and harvesting the expanded T cell, in which at least one of the activating, the transducing, and the expanding are performed in the presence of retinoic acid. The viral vector may be a retroviral vector, adenoviral vector, adeno-associated viral vector, or lentiviral vector.

In another aspect, the disclosure relates to ex-vivo methods of preparing a T cell population, activating the T cell in a fresh PBMC, e.g., the fresh PBMC is not cryopreserved, with an anti-CD3 antibody and an anti-CD28 antibody, transducing the activated T cell with a viral vector, expanding the transduced T cell, and harvesting the expanded T cell, in which at least one of the activating, the transducing, and the expanding are performed in the presence of retinoic acid.

In another aspect, each of the activating, the transducing, transfecting, or electroporating, and the expanding steps are performed in the presence of retinoic acid.

In an aspect, T cells or populations of T cells are specifically directed or homed to an organ or tissue, for example, to lung, heart, liver, pancreas, tissue, intestine, or skin by utilizing a method described herein.

In another aspect, the activating step may include immobilizing the T cell in the rested PBMC with the anti-CD3 antibody and the anti-CD28 antibody on a solid phase support.

In another aspect, the resting step may be carried out within a period of time from about 0.5 hour to about 48 hours, about 0.5 hour to about 36 hours, about 0.5 hour to about 24 hours, about 0.5 hour to about 18 hours, about 0.5 hour to about 12 hours, about 0.5 hour to about 6 hours, about 1 hour to about 6 hours, about 2 hours to about 5 hours, about 3 hours to about 5 hours, or about 1 hours to about 24 hours, about 2 to about 24 hours, about 12 to about 48 hours, about 0.5 hour to about 120 hours, about 0.5 hour to about 108 hours, about 0.5 hour to about 96 hours, about 0.5 hour to about 84 hours, about 0.5 hour to about 72 hours, or about 0.5 hour to about 60 hours.

In another aspect, the anti-CD3 antibody and the anti-CD28 antibody each have a concentration of no more than about 0.1 μg/ml, no more than about 0.2 μg/ml, no more than about 0.3 μg/ml, no more than about 0.4 μg/ml, no more than about 0.5 μg/ml, no more than about 0.6 μg/ml, no more than about 0.7 μg/ml, no more than about 0.8 μg/ml, no more than about 0.9 μg/ml, no more than about 1.0 μg/ml, no more than about 2.0 μg/ml, no more than about 4.0 μg/ml, no more than about 6.0 μg/ml, no more than about 8.0 μg/ml, or no more than about 10.0 μg/ml.

In another aspect, the anti-CD3 antibody and the anti-CD28 antibody each may have a concentration of from about 0.1 μg/ml to about 10.0 μg/ml, about 0.1 μg/ml to about 8.0 μg/ml, about 0.1 μg/ml to about 6.0 μg/ml, about 0.1 μg/ml to about 4.0 μg/ml, about 0.1 μg/ml to about 2.0 μg/ml, about 0.1 μg/ml to about 1.0 μg/ml, about 0.1 μg/ml to about 0.8 μg/ml, about 0.1 μg/ml to about 0.6 μg/ml, about 0.1 μg/ml to about 0.5 μg/ml, about 0.1 μg/ml to about 0.25 μg/ml, about 0.2 μg/ml to about 0.5 μg/ml, about 0.2 μg/ml to about 0.3 μg/ml, about 0.3 μg/ml to about 0.5 μg/ml, about 0.3 μg/ml to about 0.4 μg/ml, or about 0.4 μg/ml to about 0.5 μg/ml.

In another aspect, the activation may be carried out within a period of no more than about 1 hour, about 2 hours, about 3 hours, about 4 hours, about 5 hours, about 6 hours, about 7 hours, about 8 hours, about 9 hours, about 10 hours, about 11 hours, about 12 hours, about 14 hours, about 16 hours, about 18 hours, about 20 hours, about 22 hours, about 24 hours, about 26 hours, about 28 hours, about 30 hours, about 36 hours, about 48 hours, about 60 hours, about 72 hours, about 84 hours, about 96 hours, about 108 hours, or about 120 hours.

In another aspect, the activation may be carried out within a period of from about 1 hour to about 120 hours, about 1 hour to about 108 hours, about 1 hour to about 96 hours, about 1 hour to about 84 hours, about 1 hour to about 72 hours, about 1 hour to about 60 hours, about 1 hour to about 48 hours, about 1 hour to about 36 hours, about 1 hour to about 24 hours, about 2 hours to about 24 hours, about 4 hours to about 24 hours, about 6 hours to about 24 hours, about 8 hours to about 24 hours, about 10 hours to about 24 hours, about 12 hours to about 24 hours, about 12 hours to about 72 hours, about 24 hours to about 72 hours, about 6 hours to about 48 hours, about 24 hours to about 48 hours, about 6 hours to about 72 hours, or about 1 hours to about 12 hours.

In another aspect, the solid phase may be a surface of a bead, a plate, a flask, or a bag.

In another aspect, the plate may be a petri dish (single well), 6-well, 12-well, or 24-well plate.

In another aspect, the flask may have a seeding surface area of about 25 cm² to about 75 cm², about 25 cm² to about 100 cm², about 25 cm² to about 150 cm², or about 50 cm² to about 1720 cm².

In another aspect, the bag may have a volume of from about 5 ml to about 100 liters, about 100 ml to about 100 liters, about 150 ml to about 100 liters, about 200 ml to about 100 liters, about 250 ml to about 100 liters, about 500 ml to about 100 liters, about 1 liter to about 100 liters, about 1 liter to about 75 liters, about 1 liter to about 50 liters, about 1 liter to about 25 liters, about 1 liter to about 20 liters, about 1 liter to about 15 liters, about 1 liter to about 10 liters, about 1 liter to about 5 liters, about 1 liter to about 2.5 liters, or about 1 liter to about 2 liters.

In another aspect, the resting may be carried out in the presence of at least one cytokine. The cytokine may be interleukin 2 (IL-2), interleukin 7 (IL-7), interleukin 15 (IL-15), interleukin 21 (IL-21), or a combination thereof. The cytokine may be present in an amount at about 1 ng/mL and 500 ng/mL. The cytokine may be present in an amount of about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, or 500 ng/mL. The cytokine may be present in an amount between about 1 ng/mL and 100 ng/mL, about 100 ng/mL and 200 ng/mL, about 100 ng/mL and 500 ng/mL, about 250 ng/mL and 400 ng/mL, about 10 ng/mL and 100 ng/mL, or about 150 ng/mL and 350 ng/mL.

In another aspect, the concentration of IL-7 may be no more than about 1 ng/ml, no more than about 2 ng/ml, no more than about 3 ng/ml, no more than about 4 ng/ml, no more than about 5 ng/ml, no more than about 6 ng/ml, no more than about 7 ng/ml, no more than about 8 ng/ml, no more than about 9 ng/ml, no more than about 10 ng/ml, no more than about 11 ng/ml, no more than about 12 ng/ml, no more than about 13 ng/ml, no more than about 14 ng/ml, no more than about 15 ng/ml, no more than about 16 ng/ml, no more than about 17 ng/ml, no more than about 18 ng/ml, no more than about 19 ng/ml, no more than about 20 ng/ml, no more than about 25 ng/ml, no more than about 30 ng/ml, no more than about 35 ng/ml, no more than about 40 ng/ml, no more than about 45 ng/ml, no more than about 50 ng/ml, no more than about 60 ng/ml, no more than about 70 ng/ml, no more than about 80 ng/ml, no more than about 90 ng/ml, or no more than about 100 ng/ml.

In another aspect, the concentration of IL-7 may be from about 1 ng/ml to 90 ng/ml, about 1 ng/ml to 80 ng/ml, about 1 ng/ml to 70 ng/ml, about 1 ng/ml to 60 ng/ml, about 1 ng/ml to 50 ng/ml, about 1 ng/ml to 40 ng/ml, about 1 ng/ml to 30 ng/ml, about 1 ng/ml to 20 ng/ml, about 1 ng/ml to 15 ng/ml, about 1 ng/ml to 10 ng/ml, about 2 ng/ml to 10 ng/ml, about 4 ng/ml to 10 ng/ml, about 6 ng/ml to 10 ng/ml, or about 5 ng/ml to 10 ng/ml.

In another aspect, the concentration of IL-15 may be no more than about 5 ng/ml, no more than about 10 ng/ml, no more than about 15 ng/ml, no more than about 20 ng/ml, no more than about 25 ng/ml, no more than about 30 ng/ml, no more than about 35 ng/ml, no more than about 40 ng/ml, no more than about 45 ng/ml, no more than about 50 ng/ml, no more than about 60 ng/ml, no more than about 70 ng/ml, no more than about 80 ng/ml, no more than about 90 ng/ml, no more than about 100 ng/ml, no more than about 110 ng/ml, no more than about 120 ng/ml, no more than about 130 ng/ml, no more than about 140 ng/ml, no more than about 150 ng/ml, 200 ng/ml, 250 ng/ml, 300 ng/ml, 350 ng/ml, 400 ng/ml, 450 ng/ml, or 500 ng/ml.

In another aspect, the concentration of IL-15 may be from about 5 ng/ml to 500 ng/ml, about 5 ng/ml to 400 ng/ml, about 5 ng/ml to 300 ng/ml, about 5 ng/ml to 200 ng/ml, about 5 ng/ml to 150 ng/ml, about 5 ng/ml to 100 ng/ml, about 10 ng/ml to 100 ng/ml, about 20 ng/ml to 100 ng/ml, about 30 ng/ml to 100 ng/ml, about 40 ng/ml to 100 ng/ml, about 50 ng/ml to 100 ng/ml, about 60 ng/ml to 100 ng/ml, about 70 ng/ml to 100 ng/ml, about 80 ng/ml to 100 ng/ml, about 90 ng/ml to 100 ng/ml, about 10 ng/ml to 50 ng/ml, about 20 ng/ml to 50 ng/ml, about 30 ng/ml to 50 ng/ml, or about 40 ng/ml to 50 ng/ml.

In another aspect, the concentration of IL-2 may be from about 5 ng/ml to 500 ng/ml, about 5 ng/ml to 400 ng/ml, about 5 ng/ml to 300 ng/ml, about 5 ng/ml to 200 ng/ml, about 5 ng/ml to 150 ng/ml, about 5 ng/ml to 100 ng/ml, about 10 ng/ml to 100 ng/ml, about 20 ng/ml to 100 ng/ml, about 30 ng/ml to 100 ng/ml, about 40 ng/ml to 100 ng/ml, about 50 ng/ml to 100 ng/ml, about 60 ng/ml to 100 ng/ml, about 70 ng/ml to 100 ng/ml, about 80 ng/ml to 100 ng/ml, about 90 ng/ml to 100 ng/ml, about 10 ng/ml to 50 ng/ml, about 20 ng/ml to 50 ng/ml, about 30 ng/ml to 50 ng/ml, or about 40 ng/ml to 50 ng/ml.

In another aspect, the transducing may be carried out within a period of no more than about 1 hour, no more than about 2 hours, no more than about 3 hours, no more than about 4 hours, no more than about 5 hours, no more than about 6 hours, no more than about 7 hours, no more than about 8 hours, no more than about 9 hours, no more than about 10 hours, no more than about 11 hours, no more than about 12 hours, no more than about 14 hours, no more than about 16 hours, no more than about 18 hours, no more than about 20 hours, no more than about 22 hours, no more than about 24 hours, no more than about 26 hours, no more than about 28 hours, no more than about 30 hours, no more than about 36 hours, no more than about 42 hours, no more than about 48 hours, no more than about 54 hours, no more than about 60 hours, no more than about 66 hours, no more than about 72 hours, no more than about 84 hours, no more than about 96 hours, no more than about 108 hours, or no more than about 120 hours.

In another aspect, the transducing may be carried out within a period of from about 1 hour to 120 hours, about 1 hour to 108 hours, about 1 hour to 96 hours, about 1 hour to 72 hours, about 1 hour to 48 hours, about 1 hour to 36 hours, about 1 hour to 24 hours, about 2 hours to 24 hours, about 4 hours to 24 hours, about 6 hours to 24 hours, about 8 hours to 24 hours, about 10 hours to 24 hours, about 12 hours to 24 hours, about 14 hours to 24 hours, about 16 hours to 24 hours, about 18 hours to 24 hours, about 20 hours to 24 hours, or about 22 hours to 24 hours.

In another aspect, the viral vector may be a retroviral vector expressing a T cell receptor (TCR). The viral vector may be retroviral vector comprising a T cell receptor gene.

In another aspect, the viral vector may be a lentiviral vector expressing a TCR. The viral vector may be lentiviral vector comprising a T cell receptor gene.

In another aspect, the transducing may be carried out in the presence of at least one cytokine.

In another aspect, the at least one cytokine comprises interleukin 7 (IL-7) and/or interleukin 15 (IL-15).

In another aspect, the expanding may be carried out within a period of no more than about 1 day, no more than about 2 days, no more than about 3 days, no more than about 4 days, no more than about 5 days, no more than about 6 days, no more than about 7 days, no more than about 8 days, no more than about 9 days, no more than about 10 days, no more than about 15 days, no more than about 20 days, no more than about 25 days, or no more than about 30 days.

In another aspect, the expanding may be carried out within a period of from about 1 day to about 30 days, about 1 day to about 25 days, about 1 day to about 20 days, about 1 day to about 15 days, about 1 day to about 10 days, about 2 days to about 10 days, about 3 days to about 10 days, about 4 days to about 10 days, about 5 days to about 10 days, about 6 days to about 10 days, about 7 days to about 10 days, about 8 days to about 10 days, or about 9 days to about 10 days.

In another aspect, the number of T cells obtained by the methods of the invention may be at least about 1×10⁷, at least about 5×10⁷, at least about 1×10⁸, at least about 5×10⁸, at least about 1×10⁹, may be at least about 2×10⁹, may be at least about 3×10⁹, may be at least about 4×10⁹, may be at least about 5×10⁹, may be at least about 6×10⁹, may be at least about 7×10⁹, may be at least about 8×10⁹, may be at least about 9×10⁹, may be at least about 1×10¹⁰, may be at least about 5×10¹⁰, may be at least about 1×10¹¹, may be at least about 5×10¹¹, may be at least about 1×10¹², may be at least about 5×10¹² or may be at least about 1×10¹³ cells.

In another aspect, the number of the T cells obtained by the method of the invention may be from about 1×10⁹ to about 1×10¹³, about 1×10⁹ to about 5×10¹², about 1×10⁹ to about 1×10¹², about 1×10⁹ to about 5×10¹¹, about 1×10⁹ to about 1×10¹¹, about 1×10⁹ to about 5×10¹⁰, about 1×10⁹ to about 1×10¹⁰, about 2×10⁹ to about 1×10¹⁰, about 3×10⁹ to about 1×10¹⁰, about 4×10⁹ to about 1×10¹⁰, about 5×10⁹ to about 1×10¹⁰, about 6×10⁹ to about 1×10¹⁰, about 7×10⁹ to about 1×10¹⁰, about 8×10⁹ to about 1×10¹⁰, or about 9×10⁹ to about 1×10¹⁰ cells.

In another aspect, the T cell obtained by the method of the invention is a CD3+CD8+ T cell.

In an aspect, the present disclosure relates to a method of treating a patient having a cancer comprising administering to the patient an effective amount of the T cells produced by the methods of the present disclosure.

In an aspect, the disclose provides for methods of treating an individual or patient who has cancer, comprising administering to the individual or patient a composition comprising a population of activated T cells, and wherein said T cells are expanded and/or activated in the presence of retinoic acid; and wherein said cancer is gastrointestinal cancers, small intestine cancer, appendix cancer, anal cancer, chronic lymphocytic leukemia (CLL), acute myelogenous leukemia, bile duct cancer, brain cancer, breast cancer, colorectal carcinoma, esophageal cancer, gallbladder cancer, gastric cancer, hepatocellular cancer, Merkel cell carcinoma, melanoma, non-Hodgkin lymphoma, non-small cell lung cancer, ovarian cancer, pancreatic cancer, prostate cancer, renal cell cancer, small cell lung cancer, urinary bladder cancer, uterine cancer, or a combination thereof.

In an aspect, the present disclosure relates to T cells producible by the method of the present disclosure or compositions comprising T cells producible by the method of the present disclosure for use as a medicament.

In an aspect, the disclose provides for T cells produced by the method of the present disclosure or a compositions comprising T cells produced by the method of the present disclosure for use in the treatment of cancer, preferably said cancer is chronic lymphocytic leukemia (CLL), acute myelogenous leukemia, bile duct cancer, brain cancer, breast cancer, colorectal carcinoma, esophageal cancer, gallbladder cancer, gastric cancer, hepatocellular cancer, Merkel cell carcinoma, melanoma, non-Hodgkin lymphoma, non-small cell lung cancer, ovarian cancer, pancreatic cancer, prostate cancer, renal cell cancer, small cell lung cancer, urinary bladder cancer, uterine cancer, or a combination thereof. Preferably, said composition comprising T cells produced by the method of the present disclosure for use as a medicament, in particular for use in the treatment of cancer, in particular the herein above described cancers, comprises a population of activated T cells, and wherein said T cells are expanded and/or activated in the presence of retinoic acid.

In a further aspect, the present disclosure refers to the use of T cells produced by the method of the present disclosure or compositions comprising T cells produced by the method of the present disclosure for the manufacture of a medicament.

In a further aspect, the present disclosure refers to the use of T cells produced by the method of the present disclosure or compositions comprising T cells produced by the method of the present disclosure for the manufacture of a medicament for the treatment of cancer, in particular for the herein above mentioned cancers.

In an aspect, the activated T cells are produced by contacting T cells with the peptide loaded in complex with a human class I MHC molecule expressed on the surface of an antigen-presenting cell.

In another aspect, methods of treating an individual or patient are improved by specifically or selectively homing the T cells to an organ or tissue of interest. In an aspect, the use of a compound or composition described herein, such as retinoic acid, improves homing specificity or selectively relative to the same method without the use of a compound or composition described herein, such as retinoic acid.

In an aspect, T cells described herein are autologous to the patient or individual. In another aspect, T cells described herein are allogenic to the patient or individual.

In another aspect, the PBMC may be obtained from the patient.

In another aspect, the retinoic acid may be all-trans-retinoic acid (ATRA) or 9-cis-retinoic acid.

In another aspect, the concentration of retinoic acid may be from about 0.01 to about 10⁵ nM, from about 0.1 to about 10⁵ nM, from about 0.01 to about 10⁴ nM, from about 0.1 to about 10⁴ nM, from about 0.01 to about 10³ nM, from about 0.1 to about 10³ nM, from about 0.01 to about 100 nM, from about 0.1 to about 100 nM, from about 0.01 to about 10 nM, from about 0.1 to about 10 nM, from about 0.01 to about 1 nM, from about 0.1 to about 1 nM, from about 0.1 to about 80 nM, from about 0.01 to about 50 nM, from about 0.1 to about 20 nM, from about 0.5 to about 100 nM, from about 20 to about 100 nM, from about 10 to about 40 nM, from about 5 to about 30 nM, from about 0.01 to about 10 nM, from about 0.1 to about 10 nM, or from about 1 to about 10 nM.

In another aspect, the present disclosure relates to a genetically transduced or transfected T cell produced by the method of the present disclosure.

In an aspect, the invention refers to a substantially pure cell population of T cells, in particular genetically transduced or transfected T-cells, wherein at least 10% of the cells are CCR9+, at least 60% of the cells are α4β7+, at least 15% of the cells are CD49a+, at least 98% of the cells are CD38+, at least 20% of the cells are CD69+, and at least 60% of the cells are CD45RO+.

In another aspect, the genetically transduced T cell population may contain at least about 10%, at least about 15%, at least about 20%, at least about 25%, at least about 30%, at least about 35%, at least about 40%, from about 10% to about 15%, from about 10% to about 20%, from about 10% to about 25%, from about 10% to about 30%, from about 10% to about 35%, from about 10% to about 40%, from about 20% to about 25%, from about 20% to 30%, from about 20% to about 35%, from about 20% to about 40%, preferably from about 20% to about 30% of cells expressing CCR9 on the cell surface and at least about 60%, at least about 65%, at least about 70%, at least about 75%, at least about 80%, at least about 85%, at least about 90%, from about 60% to about 65%, from about 60% to 70%, from about 60% to about 75%, from about 60% to about 80%, from about 60% to about 85%, from about 60% to about 90%, preferably from about 60% to about 80% of cells expressing α4β7 on the cell surface.

In another aspect, the genetically transduced T cell population may contain at least about 15%, at least about 18%, at least about 20%, at least about 23%, at least about 25%, from about 15% to about 18%, from about 15% to 20%, from about 15% to about 22%, from about 15% to about 25%, from about 18% to about 20%, from about 18% to about 22%, from about 18% to about 25%, preferably from about 18% to about 22% of cells expressing CD49a on the cell surface

In another aspect, the genetically transduced T cell population may contain at least about 60%, at least about 65%, at least about 70%, at least about 75%, at least about 80%, at least about 85%, at least about 90%, at least about 95%, from about 60% to about 70%, from about 60% to 80%, from about 60% to about 90%, from about 60% to about 95%, from about 70% to about 80%, from about 70% to about 90%, from about 70% to about 95%, from about 80% to about 85%, from about 80% to about 90%, from about 80% to about 95%, preferably from about 80% to about 90% of cells expressing CD45RO on the cell surface.

In another aspect, the genetically transduced T cell population may contain at least about 98%, at least about 99%, from about 98% to about 99%, from about 98% to about 100%, from about 99% to about 100%, preferably from about 99% to about 100% of cells expressing CD38 on the cell surface.

In another aspect, the genetically transduced T cell population may contain at least about 20%, at least about 25%, at least about 30%, at least about 35%, at least about 40%, at least about 45%, at least about 50%, at least about 55%, from about 20% to about 25%, from about 20% to 30%, from about 20% to about 35%, from about 20% to about 40%, from about 20% to 45%, from about 20% to 50%, from about 20% to 55%, preferably from about 20% to about 30% of cells expressing CD69 on the cell surface.

In an aspect, the present disclosure relates to a pharmaceutical composition comprising the genetically transduced T cell of the present disclosure and a pharmaceutically acceptable carrier.

### BRIEF DESCRIPTION OF THE DRAWINGS

The patent or application file contains at least one drawing executed in color. Copies of this patent or patent application with color drawing(s) will be provided by the Office upon request and payment of the necessary fee.

FIG. 1 shows representative homing markers expressed by T cells.

FIG. 2 shows a T cell manufacturing process in accordance with one embodiment of the present disclosure.

FIGS. 3A and 3B show the effect of retinoic acid (“RA”) on T cell products in accordance with one embodiment of the present disclosure.

FIGS. 4A and 4B show the effect of retinoic acid (“RA”) on T cell products in accordance with another embodiment of the present disclosure.

FIGS. 5A and 5B show the effect of retinoic acid (“RA”) on T cell products in accordance with another embodiment of the present disclosure.

FIGS. 6A and 6B show the effect of retinoic acid (“RA”) on T cell products in accordance with another embodiment of the present disclosure.

FIG. 7 shows the effect of retinoic acid (“RA”) on T cell products in accordance with another embodiment of the present disclosure.

FIGS. 8A and 8B show the effect of retinoic acid (“RA”) on T cell products in accordance with another embodiment of the present disclosure.

FIGS. 9A and 9B show the effect of retinoic acid (“RA”) on T cell products in accordance with another embodiment of the present disclosure.

FIG. 10 shows the effect of retinoic acid (“RA”) on T cell products in accordance with another embodiment of the present disclosure.

FIGS. 11A and 11B show the effect of retinoic acid (“RA”) on T cell products in accordance with another embodiment of the present disclosure.

FIG. 12 shows the effect of retinoic acid (“RA”) on T cell products in accordance with another embodiment of the present disclosure.

FIG. 13 shows the effect of retinoic acid (“RA”) on T cell products in accordance with another embodiment of the present disclosure.

FIG. 14 shows the effect of retinoic acid (“RA”) on T cell products in accordance with another embodiment of the present disclosure.

FIG. 15 shows the effect of retinoic acid (“RA”) on T cell products in accordance with another embodiment of the present disclosure.

FIG. 16 shows the effect of retinoic acid (“RA”) on T cell products in accordance with another embodiment of the present disclosure.

FIGS. 17A and 17B show the effect of retinoic acid (“RA”) on T cell products in accordance with another embodiment of the present disclosure.

FIGS. 18A and 18B show the effect of retinoic acid (“RA”) on T cell products in accordance with another embodiment of the present disclosure.

FIGS. 19A and 19B show the effect of retinoic acid (“RA”) on T cell products in accordance with another embodiment of the present disclosure.

FIGS. 20A, 20B, and 20C show the effect of retinoic acid (“RA”) on T cell products in accordance with another embodiment of the present disclosure.

FIGS. 21A, 21B, and 21C show the effect of retinoic acid (“RA”) on T cell products in accordance with another embodiment of the present disclosure.

FIGS. 22A, 22B, and 22C show the effect of retinoic acid (“RA”) on T cell products in accordance with another embodiment of the present disclosure.

FIGS. 23A and 23B show the effect of retinoic acid (“RA”) on T cell products in accordance with another embodiment of the present disclosure.

FIGS. 24A and 24B show the killing activity of engineered T cells with or without RA in accordance with another embodiment of the present disclosure.

FIGS. 25A and 25B show the killing activity of engineered T cells with or without RA in accordance with another embodiment of the present disclosure.

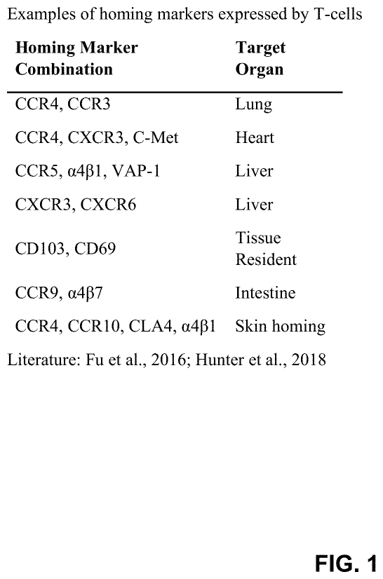

### DETAILED DESCRIPTION OF PREFERRED EMBODIMENTS

The description provides for methods of T cell homing in organs, including contacting T cells with an agonist or antagonist of a retinoic acid receptor in an amount sufficient to modulate homing of T cells to an organ. For example, the description provides for a method for genetically modifying cells comprising thawing frozen peripheral blood mononuclear cells (PBMC), resting the thawed PBMC, activating the cultured PBMC with an anti-CD3 antibody and an anti-CD28 antibody to produce an activated T cell, genetically modifying, optionally transducing, transfecting, or electroporating, the activated T cell, expanding the genetically modified, activated T cell, and obtaining the expanded T cell, wherein one or more of the activating, the genetic modification are performed in the presence of retinoic acid.

Solid tumors, if not completely metastatic, are organ specific. Targeting these organs with engineered T-cells modified to express the appropriate homing markers may lead to (1) dose sparing and faster manufacturing, (2) re-dosing of patients with cells from the same manufacturing run, (3) less adverse events due to lower doses, and (4) better and faster clearance of tumors.

Naive and effector/memory T cells have distinct repertoires of trafficking ligands and receptors that restrict their ability to interact with specialized microvessels in different anatomical compartments and, consequently, have distinct patterns of migration. “Homing of T cells to an organ” herein refers to the fact that the method of the invention leads to the expression of at least one specific marker, such as one, two, three or four markers, herein referred to as homing markers to favor their accumulation in certain target organs. The homing markers are, for example, CCR9, α4β7, CD45RO. For example, the markers CCR9 and α4β7 are mucosal homing markers, and their expression on the surface of T cells favors their homing to mucosal tissue, such as the intestine. Accordingly, since the method of the present invention increases the % of T cells expressing CCR9, α4β7, CD45RO, CD49a, CD38, and CD69 by RA treatment, an advantage of the present invention is that RA-treated engineered T cells expressing TCR that binds to tumor associated antigen/MHC complex may be used to treat certain mucosa-related cancers, such as colon cancer.

The methods described herein provide for improved T cell homing in organs including the intestines, by contacting the T cells with retinoic acid (RA). In another aspect, the RA-contacted T cell population contains higher % of CCR9+ and α4β7+ cells than that without contacting with RA.

To assess RA's ability to modulate T cell homing receptors, T cell products were manufactured in the presence or in the absence of RA, followed by receptor expression and functional analysis of the manufactured T cell products.

In an aspect, the disclosure provides for T cells populations produced by a method including thawing frozen peripheral blood mononuclear cells (PBMC), resting the thawed PBMC, activating the T cell in the rested PBMC with CD3 ligand and/or other accessory stimulation factors immobilized on a solid phase, transducing the activated T cells with a viral vector, and expanding the transduced PBMC, in which the activating, the transducing, and the expanding steps may be performed in the presence of retinoic acid.

The process for preparing a cell population containing a memory T-like cell of the present disclosure is a process may include a step of ex vivo culturing a cell population containing a T cell using retinoic acid and CD3 ligand. In accordance with the present disclosure, it is possible to prepare ex vivo a cell population containing a memory T-like cell. Further, the memory T-like cell contained in the cell population obtained by the process may have the ability to rapidly differentiate into a cell having cytotoxic activity (a cytotoxic lymphocyte) in response to even weak antigen stimulation and is suitable for utilization in an immunotherapy.

In certain aspects, the present disclosure may include a method of making and/or expanding the antigen-specific redirected T cells that comprises transfecting T cells with an expression vector containing a DNA construct encoding TCR, then, optionally, stimulating the cells with antigen positive cells, recombinant antigen, or an antibody to the receptor to cause the cells to proliferate, in which the methods are performed in the presence of RA.

In another aspect, a method is provided of stably transfecting and re-directing RA-treated T cells by electroporation, or other non-viral gene transfer (such as, but not limited to sonoporation) using naked DNA or in vitro transcribed RNA. Most investigators have used viral vectors to carry heterologous genes into T cells. By using naked DNA or RNA, the time required to produce redirected RA-treated T cells can be reduced. The electroporation method of this disclosure produces stable transfectants that express and carry on their surfaces the T-cell receptor (TCR).

Further, methods of transducing or transfecting a T cell including thawing frozen peripheral blood mononuclear cells (PBMC), resting the thawed PBMC, activating the T cell in the cultured PBMC with an anti-CD3 antibody and an anti-CD28 antibody, transducing the activated T cell with a viral vector, expanding the transduced T cell, and obtaining the expanded T cells, in which the activating, the transducing, and the expanding steps may be performed in the presence of retinoic acid are described herein.

### Definitions

Unless otherwise indicated, all terms used herein have the same meaning as they would to one skilled in the art.

“Activation,” as used herein, refers broadly refers to a state of a T cell that has been sufficiently stimulated to induce detectable cellular proliferation. In particular embodiments, activation can also be associated with induced cytokine production, and detectable effector functions. The term “activated T cells” refers to, among other things, T cells that are proliferating.

“Agonist,” as used herein, refers broadly to an agent that stimulates, increases, induces, enhances, and/or promotes an activity or expression in vitro, ex vivo or in vivo.

“Antagonist,” as used herein refers broadly to an agent that decreases, reduces, inhibits, suppresses, delays, halts, limits, controls, abrogates, eliminates, blocks, and/or prevents an activity, function or expression in vitro, ex vivo or in vivo.

“Active ingredient,” as used herein refers to retinoic acid, CD3 ligand, or mixture thereof, other accessory stimulation factors, or suitable compounds, proteins, cytokines or other components which can be contained in media used in cell culture.

“Higher” or “lower,” as used herein refers broadly to significantly higher or lower, wherein the significantly depends on the method that was used for determining the relevant value. Methods to determine a population expressing said surface marker are well known in the art and include, for example, flow cytometry. For flow cytometry a difference of more than 5%, 6%, 7%, 8%, 9%, 10%, 11%, 12% such as more than 9% of a population expressing a certain marker is considered as significantly different in the context of the invention under the tested conditions.

“Genetically modifying,” as used herein, refers broadly to the introduction of a nucleic acid into the genome of a cell. Genetically modifying includes, but is not limited to means of introduction of a nucleic acid into the genome of a cell, e.g., “transducing,” “transfecting,” and “electroporating”. The term “transduction” or “transducing” used herein generally relates to the transfer of foreign DNA or RNA into a cell by a virus or viral vector. The term “transfection,” “transfecting,” “electroporating,” or “electroporation” as used herein refer broadly to the transfer of nucleic acids by other means than transduction, e.g., plasmid transfection and RNA electroporation.

“Memory T-like cell or memory T cell,” as used herein, refers broadly to include both a central memory T-like cell and an effector memory T-like cell.

“Naked DNA,” as used herein, refers broadly to DNA encoding a TCR contained in an expression cassette or vector in proper orientation for expression.

“Peripheral blood mononuclear cell (PBMC),” as used herein, refers broadly to any blood cell with a round nucleus (e.g., a lymphocyte, a monocyte, or a macrophage). These blood cells are a critical component in the immune system to fight infection and adapt to intruders. The lymphocyte population consists of CD4+ and CD8+ T cells, B cells and Natural Killer cells, CD14+ monocytes, and basophils/neutrophils/eosinophils/dendritic cells. These cells are often separated from whole blood or from leukopacks using FICOLL™ (a hydrophilic polysaccharide) that separates layers of blood, with monocytes and lymphocytes forming a buffy coat under a layer of plasma. In one embodiment, “PBMCs” may refer to a population of cells comprising at least T cells, and optionally NK cells, and antigen presenting cells.

“Retinoic acid (RA),” as used herein, refers broadly to the bioactive metabolite of Vitamin A (retinol). Retinoic acid includes, but is not limited to, all-trans-retinoic acid (ATRA), in which all double bonds on the chain part are in transform, or 9-cis-retinoic acid, in which a double bond at the 9-position is cis form. Other retinoic acid isomers and retinoic acid derivatives can be also used in the methods and compositions described herein. Retinoic acids, retinoic acid isomers and retinoic acid derivatives or their salts may be collectively referred to as a “retinoic acid”. In the present disclosure, retinoic acid used herein may be one kind of retinoic acid or a combination of different kinds of retinoic acid.

“RNA,” as used herein, refers broadly to in vitro transcribed RNA that can be translated into a protein, e.g., TCR, in cytoplasm.

“T cell” or “T lymphocyte,” as used herein, refers broadly to thymocytes, naïve T lymphocytes, immature T lymphocytes, mature T lymphocytes, resting T lymphocytes, or activated T lymphocytes.

“T-cell receptor (TCR),” as used herein, refers broadly to a protein receptor on T cells that is composed of a heterodimer of an alpha (α) and beta (β) chain, although in some cells the TCR consists of gamma and delta (γ/δ) chains.

“Unit dosage form,” as used herein, refers broadly to physically discrete units suitable as unitary dosages for human and animal subjects, each unit containing a predetermined quantity of the composition of the present invention, alone or in combination with other active agents, calculated in an amount sufficient to produce the desired effect, in association with a pharmaceutically acceptable diluent, carrier, or vehicle, where appropriate.

Activation of T-Cells with Retinoic Acid

In certain aspects, the T cells are primary human T cells, such as T cells derived from human peripheral blood mononuclear cells (PBMC), PBMC collected after stimulation with G-CSF, bone marrow, or umbilical cord blood. Conditions include the use of mRNA and DNA and electroporation. Following transfection, cells may be immediately infused or may be stored. In certain aspects, following transfection, the cells may be propagated for days, weeks, or months ex vivo as a bulk population within about 1, 2, 3, 4, 5 days or more following gene transfer into cells. In a further aspect, following transfection, the transfectants are cloned and a clone demonstrating presence of a single integrated or episomally maintained expression cassette or plasmid, and expression of the TCR is expanded ex vivo. The clone selected for expansion demonstrates the capacity to specifically recognize and lyse peptide-expressing target cells. The recombinant T cells may be expanded by stimulation with IL-2, or other cytokines that bind the common gamma-chain (e.g., IL-7, IL-12, IL-15, IL-21, and others). The recombinant T cells may be expanded by stimulation with artificial antigen presenting cells. The recombinant T cells may be expanded on artificial antigen presenting cell or with an antibody, such as OKT3, which cross links CD3 on the T cell surface. Subsets of the recombinant T cells may be deleted on artificial antigen presenting cell or with an antibody, such as CAMPATH® (monoclonal anti-CD52 antibody), which binds CD52 on the T cell surface. In a further aspect, the genetically modified cells may be cryopreserved.

### Culture Conditions

Although a medium not containing serum or plasma may be used in the present disclosure, serum or plasma may be added to the medium. The amount of serum or plasma to be added to the medium is not particularly limited, and the content of serum or plasma in the medium may be, for example, more than 0 to 20% by volume, preferably more than 0 to 5% by volume. The amount of serum or plasma to be used can be changed depending on the culturing stage. For example, serum or plasma can also be used while step wise decreasing the concentration thereof. The serum or plasma may be self-derived (meaning that the origin is the same as that of a cell to be cultured) or non-self-derived (meaning that the origin is different from that of a cell to be cultured). From the viewpoint of safety, self-derived serum or plasma may be used.

The cell number at initiation of culture used in the present disclosure may be not particularly limited, and may be, for example, from about 10 cells/ml to about 1×10⁸ cells/ml, from about 100 cells/ml to about 5×10⁷ cells/ml, from about 1000 cells/ml to about 2×10⁷ cells/ml, from about 1000 cells/ml to about 5×10⁶ cells/ml, and from about 1000 cells/ml to about 2×10⁶ cells/ml. The culture conditions are not particularly limited, and conditions which are usually used for cell culture can be used. For example, cells can be cultured under the conditions of 37° C. and 5% CO₂. An additional operation, such as adding a fresh medium to a cell culture liquid at a suitable time interval to dilute the liquid, exchanging a medium, or exchanging a cell culture instrument may be carried out.

The cell culture instrument used in the process for preparing a cell population of the present disclosure may be not particularly limited, and examples thereof may include a petri dish, a flask, a bag, a large culture tank, and a bioreactor. As the bag, a CO₂ gas-permeable cell culture bag can be used. In the case of industrially preparing a large amount of a cell population, a large culture tank can be used. Although cell culture can be carried out in either an open system, semi-closed, or a closed system, it may be preferable to carry out the cell culture in a closed system from the viewpoint of safety of the obtained cell population.

Retinoic acid may be added to a cell culture liquid containing T cells or precursor cells of T cells from the initiation of culture. For example, culturing step in the presence of a retinoic acid and a CD3 ligand may be carried out for at least 1 day or longer, more preferably 2 to 7 days, further preferably 2 to 5 days from the initiation of culture. Since there is a possibility that a retinoic acid is degraded in a culture liquid, a retinoic acid may be newly added at a suitable time interval.

Culture conditions may be not particularly limited, and conditions, which are usually used for cell culture can be used. For example, cells can be cultured under the conditions of 37° C. and 5% CO₂. In addition, a medium can be exchanged with a fresh medium at a suitable time interval.

In the process of the present disclosure, culturing the memory T-like cell may be usually carried out in a medium containing predetermined components in the presence of active ingredients of the present disclosure. The cell number at initiation of culture used in the present invention may be not particularly limited, and may be, for example, from about 10 cells/ml to about 1×10⁸ cells/ml, from about 100 cells/ml to about 5×10⁷ cells/ml, from about 1000 cells/ml to about 2×10⁷ cells/ml, from about 1000 cells/ml to about 5×10⁶ cells/ml, and from about 1000 cells/ml to about 2×10⁶ cells/ml.

The number of days of culture may be from 4 to 14 days. For example, the number of days in culture may be from 4 to 14 days, from 4 to 13 days, from 4 to 12 days, from 4 to 11 days, from 4 to 10 days, from 4 to 9 days, from 4 to 8 days, from 4 to 7 days, from 4 to 6 days, and from 4 to 5 days. The number of days in culture may be about 4, 5, 6, 7, 8, 9, 10, 11, 12, 13, or 14 days.

### Cell Population

Cell population obtained by the process of the present disclosure may contain a high proportion of cells, which may not express CD45RA, but express CCR7 and/or CD62L, and are negative for both CCR7 and CD62L. All of CD45RA, CCR7 and CD62L are cell surface antigen markers of a lymphocyte. That is, cells contained in the cell population obtained by the process of the present disclosure at a high proportion may be classified into a memory T-like cell. Memory T cells generally exhibit a high survival rate in a living body upon administration to the living body, a great cell proliferating effect, a great effect of accumulation into a tumor, and a high production rate of tumor-specific effector cells, and therefore they may be useful in the field of cell therapy. In other words, the process of the present disclosure can increase the proportion of a memory T-like cell in a cell population. As used herein, an increase in the proportion of a memory T-like cell means that, when culture is carried out under the same conditions except for the presence or absence of the active ingredients of the present invention, the proportion of a memory T-like cell in a cell population obtained by carrying out the culture in the presence of the active ingredients used in the process of the present invention is higher as compared with the culture in the absence of the active ingredients. Preferably, a cell population having the proportion of a memory T-like cell which is 5% or more, more preferably 10% or more higher than that of a cell population obtained in the absence of the active ingredients can be obtained. In addition, the cell population obtained by the process of the present invention contains Tc2 type- and/or Th2 type-phenotype cells at a high proportion.

Cell population obtained by the process of the present disclosure may be further cultured in a medium containing or not containing retinoic acid in the absence of CD3 ligand, e.g., anti-CD3 antibody and/or other accessory stimulation factors, e.g., anti-CD28 antibody.

In the cell population obtained by the process for preparing a cell population containing a memory T-like cell of the present disclosure, usually, cells other than the memory T-like cell may be also present. In the present disclosure, cells may be collected from the cell population by centrifugation or the like and can be used as the memory T-like cell obtained by the process of the present disclosure, for example, as they are. If the active ingredients and the like are immobilized onto a cell culture instrument, commingling of the active ingredients and the like with the resulting memory T-like cell can be prevented.

The process of the present disclosure may further comprise a step of separating the memory T-like cell from the cell population. That is, in the present disclosure, a cell population in which the memory T-like cell may be concentrated can be prepared for use by carrying out an operation of separating the cell population containing the memory T-like cell into cells other than the memory T-like cell and the memory T-like cell. For example, the process may comprise a step of removing CD45RA positive cells. Separation of the memory T-like cell can be carried out according to known methods. For example, the memory T-like cell can be separated by selectively collecting CD3 positive and CD45RA negative cells, which can be distinguished using a flow cytometer by co-staining with a fluorescently labeled anti-CD3 antibody and a fluorescently labeled anti-CD45RA antibody. Alternatively, a cell population containing a high proportion of a memory T-like cell can be obtained by removing cells other than the memory T-like cell from the cell population obtained by the process of the present disclosure. The cell population containing a high proportion of a memory T-like cell according to the present disclosure may also include a cell population containing only the memory T-like cell.

### Retinoic Acid and the Retinoic Acid Receptor

Vitamin A and its active metabolite retinoic acid (RA) are essential for the development and function of many tissues including the immune system. Upon transportation into the nucleus, RA binds to retinoic acid receptor (RAR)/retinoic X receptor (RXR) heterodimers and these complexes regulate transcriptional activity of target genes via binding to retinoic acid response elements (RAREs) in the promoter regions.

The retinoid signal may be transduced by two families of nuclear receptors, e.g., the retinoic acid receptor (RAR) family containing three isotypes, RARα, RARβ, and RARγ, and the retinoid X receptor (RXR) family containing also three isotypes, RXRα, RXRβ, and RXRγ. Each RAR and RXR isotype includes several isoforms. These receptors belong to the superfamily of nuclear hormone receptors and act as ligand-activated transcription factors. RARs function as a heterodimer together with RXR. The ligand-receptor complexes act as inducible transcription regulators of several genes by binding to specific retinoic acid response elements (RARE), e.g., the DR-2 type and the DR-5 type, located on retinoic acid-regulated genes. The RXR can also act as a homodimer on transcription activation via the retinoid X response element (DR-1) and as a heterodimer with several nuclear receptors, e.g., the thyroid hormone receptor (TR), the peroxisome-proliferator activated receptor, and the vitamin D receptor, on their specific response elements. The natural ligands for the RARs are ATRA and its stereoisomers 9-cis-RA and 13-cis-RA, whereas RXRs are activated by 9-cis-RA.

The morphogenic role of the active form of vitamin A, retinoic acid (RA), in controlling spatial and temporal developmental patterning has underscored the powerful and essential function of this mediator during embryogenesis. Similarly, within the immune system, RA has been shown to exert profound effects as a differentiation factor in inducing gut homing of leukocytes, the differentiation and stability of adaptive regulatory T cells, the differentiation of CD4⁺ T cells toward T-helper 1(T_{H}1)/T_{H}17 cells, IgA class switching in B cells, and the differentiation of myeloid cells. RA function is also essential for the survival of tumor-reactive CD8⁺ T cells within the tumor microenvironment. T cells express RARs and are major targets of RA regulation. RAs and their receptors appear to regulate T cells through genomic and non-genomic functions. RAs may affect, among other things, gut-homing receptor expression. For example, in the intestine, RAs may promote gut-homing effector T cells (Th1 and Th17).

Retinoic acid receptor agonists include any molecule that activates, stimulates induces, enhances or promotes a retinoic acid receptor activity or function in vitro, ex vivo, or in vivo. Non-limiting examples of retinoic acid receptor agonists applicable in the compositions and methods include vitamin A, and vitamin A derivatives, analogues and metabolites. Non-limiting examples of vitamin A metabolites include retinoic acid (RA), and retinoic acid derivatives, analogues and isomers. Non-limiting examples of retinoic acid derivatives include an esters and amides, such as fenretinide and retinaldehyde. Non-limiting examples of retinoic acid analogues include 9-cis retinoic acid, 13-cis retinoic acid and all trans retinoic acid (ATRA). Non-limiting examples of ATRA metabolites include 4-hydroxy-retinoic acid (4-OH-RA), 4-oxo-retinoic acid (4-oxo-RA), 18-hydroxy-retinoic acid (18-OH-RA), and 5,6-epoxy-retinoic acid (5,6-epoxy-RA). Non-limiting examples of retinoic acid isomers include an arotinoid, such as adapalene and tazarotene.

The concentration of retinoic acid used in the medium may be not particularly limited, for example, from about 0.01 to about 10⁵ nM, from about 0.1 to about 10⁵ nM, from about 0.01 to about 10⁴ nM, from about 0.1 to about 10⁴ nM, from about 0.01 to about 10³ nM, from about 0.1 to about 10³ nM, from about 0.01 to about 100 nM, from about 0.1 to about 100 nM, from about 0.01 to about 10 nM, from about 0.1 to about 10 nM, from about 0.01 to about 1 nM, from about 0.1 to about 1 nM, from about 0.1 to about 80 nM, from about 0.01 to about 50 nM, from about 0.1 to about 20 nM, from about 0.5 to about 100 nM, from about 20 to about 100 nM, from about 10 to about 40 nM, from about 5 to about 30 nM, from about 0.01 to about 10 nM, from about 0.1 to about 10 nM, or from about 1 to about 10 nM.

### T Cells

A T cell is also called a T lymphocyte, and means a cell derived from a thymus among lymphocytes involved in an immunological response. The T cell may include a differentiated T cell and an undifferentiated T cell. Examples of known T cell include a helper T cell, a suppressor T cell, a killer T cell, a naive T cell, a memory T cell, an αβ T cell expressing TCRs of an α chain and a β chain, and a γδ T cell expressing TCRs of a γ chain and a δ chain. As used herein, examples of the “cell population containing a T cell or a precursor cell of a T cell” include, but not particularly limited to, a peripheral blood mononuclear cell (PBMC), a naive T cell, a hematopoietic stem cell, and an umbilical blood mononuclear cell. A variety of cell populations derived from hemocyte cells containing a T cell can be also used in the present invention. These cells may be activated in vivo or ex vivo by a cytokine, such as IL-2. These cells may be collected from a living body or obtained through culturing ex vivo, and then may be used as they are or after freezing preservation. For example, cell populations obtained through various derivation operations or separation operations from cell populations obtained from a living body, for example, any cell populations obtained by separating cells such as a PBMC into CD8+(positive) or CD4+(positive) cells can be also used. Further, in the process for preparing a cell population of the present disclosure, materials containing the above-mentioned cells, for example, blood, such as peripheral blood and umbilical blood, or materials obtained by removing components, such as erythrocytes or plasma from blood, and bone marrow fluid can also be used. T cells for use in the therapeutic methods described herein may be obtained from PB MC.

T cells may be isolated from leukapheresis product, e.g., LeukoPak® (enriched leukapheresis product), of a subject, for example, a human subject. T cells may be not isolated from peripheral blood mononuclear cells (PBMC), such as cord blood. The blood sample may comprise peripheral blood mononuclear cells (PBMC) and/or leukapheresis product.

Illustrative populations of T cells suitable for use in particular embodiments include, but are not limited to, helper T cells (HTL; CD4+ T cell), a cytotoxic T cell (CTL; CD8+ T cell), CD4+CD8+ T cell, CD4−CD8− T cell, or any other subset of T cells. Other illustrative populations of T cells suitable for use in particular embodiments include, but are not limited to, T cells expressing one or more of the following markers: CD3, CD4, CD8, CD27, CD28, CD45RA, CD45RO, CD62L, CD127, CD197, and HLA-DR and if desired, can be further isolated by positive or negative selection techniques.

A memory T cell is a specific type of a T cell capable of recognizing a foreign invader, such as a bacterium or a virus, which has been previously encountered via infection or vaccination. Upon a second encounter with an invader, the memory T cell initiates an immunological response faster and stronger than the time when the immune system first responded to the invader. The memory T cell may include two different cell populations of a central memory T cell and an effector memory T cell, based on difference in the homing ability or the effector function. The central memory T cell is believed to exhibit the property of a memory stem cell and may have the ability to self-replicate by high level phosphorylation of an important transcription factor known as STATS. The central memory T cell is negative for CD45RA but is positive for both CCR7 and L-selectin (CD62L). The effector memory T cell is negative for CCR7 and CD62L in addition to CD45RA.

The memory T cell is distinguished from a naive T cell by analysis of its cell surface antigen. The naive T cell is positive for cell surface antigen markers CD45RA, CCR7 and CD62L, while the memory T cell consisting of the central memory T cell and the effector memory T cell is negative for CD45RA. The memory T cell can be also distinguished from the naive T cell in that the memory T cell can rapidly initiate an immunological response as described above.

In the present disclosure, preparation of a cell population containing a memory T-like cell means a concept including induction of a memory T-like cell from a precursor cell having an ability to differentiate into a memory T-like cell, and proliferation (expansion culture) of the memory T-like cell. According to the present disclosure, a cell population containing a high proportion of a memory T-like cell may be obtained.

The T-cells may be activated, where the T cell that has been sufficiently stimulated to induce detectable cellular proliferation. In particular embodiments, activation can also be associated with induced cytokine production, and detectable effector functions. Additionally, activated T cells, among other things, are proliferating. Signals generated through the TCR alone are insufficient for full activation of the T cell and one or more secondary or costimulatory signals are also required. Thus, T cell activation comprises a primary stimulation signal through the TCR/CD3 complex and one or more secondary costimulatory signals. Costimulation can be evidenced by proliferation and/or cytokine production by T cells that have received a primary activation signal, such as stimulation through the CD3/TCR complex or through CD2.

A resting T cell generally refers to a T cell that is not actively dividing or producing cytokines. Resting T cells are small (approximately 6-8 microns) in size compared to activated T cells (approximately 12-15 microns).

A primed T cell generally refers to a resting T cell that has been previously activated at least once and has been removed from the activation stimulus for at least about 1 hour, at least about 2 hours, at least about 3 hours, at least about 4 hours, at least about 5 hours, at least about 6 hours, at least about 12 hours, at least about 24 hours, at least about 48 hours, at least about 60 hours, at least about 72 hours, at least about 84 hours, at least about 96 hours, at least about 108 hours, or at least about 120 hours. For example, a primed T cell may be a resting T cell that has been previously activated at least once and has since been removed from the activation stimulus for between about 1 to 120 hours. This time may be referred to as “resting.” Alternatively, resting may be carried out within a period of from about 0.5 hour to about 120 hours, about 0.5 hour to about 108 hours, about 0.5 hour to about 96 hours, about 0.5 hour to about 84 hours, about 0.5 hour to about 72 hours, about 0.5 hour to about 60 hours, about 0.5 hour to about 48 hours, about 0.5 hour to about 36 hours, about 0.5 hour to about 24 hours, about 0.5 hour to about 18 hours, about 0.5 hour to about 12 hours, about 0.5 hour to about 6 hours, about 1 hour to about 6 hours, about 2 hours to about 5 hours, about 3 hours to about 5 hours, or about 4 hours to about 5 hours. Primed T cells usually have a memory phenotype.

### CD2, CD3, and CD28 Antigens and Antibodies

In immunology, the CD3 antigen (CD stands for cluster of differentiation) is a protein complex composed of four distinct chains (CD3-γ, CD3δ, and two times CD3ε) in mammals, that associate with molecules known as the T-cell receptor (TCR) and the ζ-chain to generate an activation signal in T lymphocytes. The TCR, ζ-chain, and CD3 molecules together comprise the TCR complex. The CD3-γ, CD3δ, and CD3ε chains are highly related cell surface proteins of the immunoglobulin superfamily containing a single extracellular immunoglobulin domain. The transmembrane region of the CD3 chains is negatively charged, a characteristic that allows these chains to associate with the positively charged TCR chains (TCRα and TCRβ). The intracellular tails of the CD3 molecules contain a single conserved motif known as an immunoreceptor tyrosine-based activation motif or ITAM for short, which is essential for the signaling capacity of the TCR.

CD3 ligand and CD2 ligand used in the present disclosure as the active ingredient may be not particularly limited as long as it is a substance having the activity of binding to CD3 and CD2. Examples of the CD3 ligand include an anti-CD3 antibody, ConA, PHA, and PMA+ionomycin. Anti-CD3 antibodies are known in the art and are commercially available. See, e.g., U.S. Pat. Nos. 5,929,212; 6,750,325; 8,551,478; 9,650,445; 10,046,008; Particularly preferably, an anti-CD3 monoclonal antibody, for example, OKT3 may be used in the present disclosure. Examples of the CD2 ligand may include anti-CD2 antibody, CD58 (LFA-3), and CD59. The concentration of the CD3 ligand and the CD2 ligand in the medium may be not particularly limited. For example, when an anti-CD3 monoclonal antibody or an anti-CD2 monoclonal antibody is used, the concentration may be from about 0.001 to about 100 μg/mL, from about 0.01 to about 100 μg/mL, from about 0.1 to about 100 μg/mL, from about 1 to about 100 μg/mL, from about 5 to about 100 μg/mL, from about 10 to about 100 μg/mL, from about 20 to about 100 μg/mL, from about 30 to about 100 μg/mL, from about 40 to about 100 μg/mL. from about 50 to about 100 μg/mL, from about 60 to about 100 μg/mL, from about 70 to about 100 μg/mL, from about 80 to about 100 μg/mL, from about 90 to about 100 μg/mL, and from about 95 to about 100 μg/mL. For the purpose of activating a receptor on a lymphocyte, an anti-CD3 antibody may be added to the medium.

CD28 is one of the molecules expressed on T cells that provide co-stimulatory signals, which are required for T cell activation. CD28 is the receptor for B7.1 (CD80) and B7.2 (CD86). When activated by Toll-like receptor ligands, the B7.1 expression is upregulated in antigen presenting cells (APCs). The B7.2 expression on antigen presenting cells is constitutive. CD28 is the only B7 receptor constitutively expressed on naive T cells. Stimulation through CD28 in addition to the TCR can provide a potent co-stimulatory signal to T cells for the production of various interleukins (IL-2 and IL-6 in particular). In the present disclosure, if necessary, accessory stimulation can be also introduced by adding other accessory stimulation factors, such as a CD28 ligand, e.g., anti-CD28 antibody. Anti-CD28 antibodies are known in the art and are commercially available. See, e.g., U.S. Pat. Nos. 10,434,120; 10,239,931; 8,785,604; and 6,887,466. For example, when an anti-CD28 monoclonal antibody is used, the concentration may be from about 0.001 to about 100 μg/mL, from about 0.01 to about 100 μg/mL, from about 0.1 to about 100 μg/mL, from about 1 to about 100 μg/mL, from about 5 to about 100 μg/mL, from about 10 to about 100 μg/mL, from about 20 to about 100 μg/mL, from about 30 to about 100 μg/mL, from about 40 to about 100 μg/mL. from about 50 to about 100 μg/mL, from about 60 to about 100 μg/mL, from about 70 to about 100 μg/mL, from about 80 to about 100 μg/mL, from about 90 to about 100 μg/mL, and from about 20 to about 500 μg/mL. Examples of other accessory stimulation factors may include a desired antigen, a glucocorticoid-induced TNF-related receptor ligand (GITRL), an anti-CD28 antibody, an anti-CD80 antibody, and an anti-CD86 antibody. Anti-CD80 and anti-CD86 antibodies are known in the art and are commercially available. See, e.g., U.S. Pat. Nos. 8,969,531 and 8,378,082.

Among these components, CD3 ligand, e.g., anti-CD3 antibody, and the other accessory stimulation factors, e.g., anti-CD28 antibody, may be dissolved in the medium to make them coexist or may be immobilized onto a suitable solid phase, for example, an instrument for cell culture (including an open system and a closed system), such as a petri dish, a flask or a bag; or a support for cell culture, such as beads, a membrane or a glass slide, when they are used. Materials of the solid phase may be not particularly limited as long as they can be used for cell culture. For example, in the case where the components are immobilized onto the instrument, it is preferable that a certain amount of each component relative to the amount of the medium that will be put in the instrument is immobilized so that when the medium is put into the instrument, the ratio of the component to the medium is the same as the desired concentration for the case of dissolving the component in the medium. However, the amounts of immobilization of the components may be not particularly limited as long as the desired effect is obtained. When the support is used, it is immersed in a culture liquid in an instrument for cell culture during cell culture. In the case where the components are immobilized onto the support, it is preferable that a certain amount of each component relative to the amount of the medium that will be put into the instrument is immobilized so that when the support is put into the medium, the ratio of the component to the medium is the same as the desired concentration for the case of dissolving the component in the medium. However, the immobilization amounts of the components may be not particularly limited as long as the desired effect is obtained. Further, an active ingredient other than the CD3 ligand and the other accessory stimulation factors may be immobilized onto an instrument for cell culture or a support for cell culture.

If a component selected from the above-mentioned various components and the active ingredients of the present disclosure is immobilized onto a solid phase, the memory T-like cells can be easily separated from the other components and the like by simply separating the memory T-like cells from the solid phase after the memory T-like cells are obtained by the process of the present disclosure, and thereby commingling of the other components and the like with the memory T-like cells can be prevented.

### Cytokines

The steps of resting, activation, and/or expansion of the T cells may be carried out in the presence of at least one cytokine. The cytokine may be IL-2, IL-7, IL-12, IL-15, IL-21, or combination thereof. The cytokine may be present in an amount at about 1 ng/mL and 500 ng/mL.

The cytokine may be present in an amount of about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, or 500 ng/mL.

The cytokine is present in an amount between about 1 ng/mL and 100 ng/mL, about 100 ng/mL and 200 ng/mL, about 100 ng/mL and 500 ng/mL, about 250 ng/mL and 400 ng/mL, about 10 ng/mL and 100 ng/mL, or about 150 ng/mL and 350 ng/mL.

The amount of IL-2 may be between about 5 ng/mL and 500 ng/mL.

The amount of IL-7 may be between about 1 ng/mL and 100 ng/mL.

The amount of IL-15 may be between about 5 ng/mL and 500 ng/mL.

The concentration of the cytokines in the medium may be not particularly limited, for example, from about 0.1 to about 200 ng/ml, from about 0.1 to about 150 ng/ml, from about 0.1 to about 100 ng/ml, from about 1 to about 100 ng/ml, from about 2 to about 100 ng/ml, from about 4 to about 100 ng/ml, from about 6 to about 100 ng/ml, from about 8 to about 100 ng/ml, from about 10 to about 100 ng/ml, from about 20 to about 100 ng/ml, from about 30 to about 100 ng/ml, from about 40 to about 100 ng/ml, from about 50 to about 100 ng/ml, from about 60 to about 100 ng/ml, from about 70 to about 100 ng/ml, from about 80 to about 100 ng/ml, and from about 90 to about 100 ng/ml. In addition, a lymphocyte stimulation factor, such as lectin, may also be added to the medium.

### Genetic Modification

The activated T cells may be genetically modified by means of introducing a nucleic acid into their genome, for example, by transducing, transfecting, or electroporating the activated T cells. Methods of genetic modification of cells are known in the art. See, e.g., Molecular Cloning: A Laboratory Manual (4^{th} Ed.) Cold Spring Harbor Laboratory Press.

In the process for preparing a cell population, into which a gene is transferred of the present disclosure, the activating step may be carried out in the presence of RA to prepare cell population containing memory T-like cells. After culturing the activated T cells for at least 1 day or longer, e.g., 1 day, 1 to 7 days, 1 to 6 days, 1 to 5 days, 1 to 4 days, 1 to 3 days, or 1 to 2 days, followed by transducing the activated T cells. For example, the activated T cells may be cultured for 1, 2, 3, 5, 6, or 7 days.

The number of the desired gene to be transferred into the cell may be not limited, and one gene or plural genes (e.g., 1 to 9 genes) may be transferred. For example, a suitable gene such as a gene encoding a T cell surface antigen can be transferred at the same time, in advance, or afterwards, depending on the cell into which the gene is transferred. For example, in the case where an αβ TCR gene is transferred into a γδ T cell, it is preferable that a gene encoding CD8 is transferred simultaneously.

The desired gene to be transferred into a T cell in the present disclosure may be not particularly limited, and an arbitrary gene which is desired to be transferred into the cell can be selected from self-derived genes and foreign genes. Examples of such a gene may include a gene encoding an antisense nucleic acid, a siRNA (small interfering RNA) or a ribozyme as well as a gene encoding a protein (e.g., an enzyme, a cytokine, or a receptor). In addition, a suitable marker gene which enables gene-transferred cells to be selected may be transferred together with the above-mentioned genes.

The desired gene to be transferred may be obtained from nature, or may be prepared by genetic engineering procedure, or may prepared by binding DNA molecules from different origins via a known means such as ligation. Further, the desired gene may have a sequence in which a mutation is introduced into the original sequence depending on the purpose.

According to the process of the present disclosure, for example, a gene encoding an enzyme associated with resistance to a drug used in treatment of a patient with cancer or the like can be transferred into a lymphocyte to confer the drug resistance to the lymphocyte. When the lymphocyte is used, an adoptive immunotherapy and a drug therapy can be combined, and thereby a higher therapeutic effect can be obtained. An example of the drug resistance gene is a multidrug resistance gene. On the other hand, contrary to the above-mentioned aspect, a gene conferring sensitivity to a specific drug can be transferred into a lymphocyte to confer sensitivity to the drug. In such a case, it becomes possible to remove a lymphocyte after transplantation into a living body by administering the drug. An example of the gene conferring sensitivity to a drug is a thymidine kinase gene.

One aspect of the present disclosure is exemplified by transfer of a gene encoding a receptor which recognizes a desired antigen, without particular limitation. Examples of the gene include a gene encoding a T cell receptor (TCR) which recognizes a surface antigen of a target cell, and a gene which has an antigen recognition site of an antibody to a surface antigen of a target cell and encodes a chimeric receptor comprising an intracellular region of a TCR. A cell population containing a T cell into which the gene is transferred is a cell population containing a T cell which recognizes the desired antigen. Since the cell population may have higher specificity for a desired antigen as compared with a cell population into which a gene encoding a receptor is not transferred and can specifically react with the desired antigen in response to stimulation by the desired antigen, it is useful for utilization in immunotherapy.

The T-cell receptor (TCR) is a molecule found on the surface of T lymphocytes (or T cells) that is generally responsible for recognizing antigens bound to major histocompatibility complex (MHC) molecules. It is a heterodimer consisting of an alpha and beta chain in 95% of T cells, while 5% of T cells have TCRs consisting of gamma and delta chains. Engagement of the TCR with antigen and MHC results in activation of its T lymphocyte through a series of biochemical events mediated by associated enzymes, co-receptors, and specialized accessory molecules.

T-cell based immunotherapy targets peptide epitopes derived from tumor-associated or tumor-specific proteins, which are presented by molecules of the major histocompatibility complex (MHC). The antigens that are recognized by the tumor specific T lymphocytes, that is, the epitopes thereof, can be molecules derived from all protein classes, such as enzymes, receptors, transcription factors, etc. which are expressed and, as compared to unaltered cells of the same origin, usually upregulated in cells of the respective tumor.

There are two classes of MHC-molecules, MHC class I and MHC class II. MHC class I molecules are composed of an alpha heavy chain and beta-2-microglobulin, MHC class II molecules of an alpha and a beta chain. Their three-dimensional conformation results in a binding groove, which is used for non-covalent interaction with peptides. MHC class I molecules can be found on most nucleated cells. They present peptides that result from proteolytic cleavage of predominantly endogenous proteins, defective ribosomal products (DRIPs) and larger peptides. However, peptides derived from endosomal compartments or exogenous sources are also frequently found on MHC class I molecules. This non-classical way of class I presentation is referred to as cross-presentation. MHC class II molecules can be found predominantly on professional antigen presenting cells (APCs), and primarily present peptides of exogenous or transmembrane proteins that are taken up by APCs e.g., during endocytosis, and are subsequently processed.

Complexes of peptide and MHC class I are recognized by CD8-positive T-cells bearing the appropriate T-cell receptor (TCR), whereas complexes of peptide and MHC class II molecules are recognized by CD4-positive-helper-T-cells bearing the appropriate TCR. It is well known that the TCR, the peptide and the MHC are thereby present in a stoichiometric amount of 1:1:1.

TCR constructs of the present disclosure may be applicable in subjects having or suspected of having cancer by reducing the size of a tumor or preventing the growth or re-growth of a tumor in these subjects. Accordingly, the present disclosure further relates to a method for reducing growth or preventing tumor formation in a subject by introducing a TCR construct of the present disclosure into an isolated T cell of the subject and reintroducing into the subject the transformed T cell, thereby effecting anti-tumor responses to reduce or eliminate tumors in the subject. Suitable T cells that can be used include cytotoxic lymphocytes (CTL) or any cell having a T cell receptor in need of disruption. As is well-known to one of skill in the art, various methods are readily available for isolating these cells from a subject. For example, using cell surface marker expression or using commercially available kits (e.g., ISOCELL™ from Pierce, Rockford, Ill.)

It is contemplated that the TCR construct can be introduced into the subject's own T cells as naked DNA in a suitable vector or in vitro transcribed RNA. Methods of stably transfecting T cells by electroporation using naked DNA or RNA are known in the art. See, e.g., U.S. Pat. No. 6,410,319. Naked DNA generally refers to the DNA encoding a TCR of the present disclosure contained in a plasmid expression vector in proper orientation for expression. RNA generally refers to in vitro transcribed RNA that can be translated into a protein, e.g., TCR, in cytoplasm. Advantageously, the use of naked DNA or RNA reduces the time required to produce T cells expressing the TCR of the present disclosure.

In embodiments of the disclosure, the T-cell receptor (TCR) may be modified on any cell comprising a TCR, including a helper T cell, a cytotoxic T cell, a memory T cell, regulatory T cell, natural killer T cell, and γδ T cell, for example.

Alternatively, a viral vector (e.g., a retroviral vector, adenoviral vector, adeno-associated viral vector, or lentiviral vector) can be used to introduce the TCR construct into T cells. Suitable vectors for use in accordance with the method of the present disclosure are non-replicating in the subject's T cells. A large number of vectors are known that are based on viruses, where the copy number of the virus maintained in the cell is low enough to maintain the viability of the cell. Illustrative vectors include the pFB-neo vectors (STRATAGENE®) as well as vectors based on HIV, SV40, EBV, HSV, or BPV.

Once it is established that the transfected or transduced T cell is capable of expressing the TCR construct as a surface membrane protein with the desired regulation and at a desired level, it can be determined whether the TCR is functional in the host cell to provide for the desired signal induction. Subsequently, the transduced T cells are reintroduced or administered to the subject to activate anti-tumor responses in the subject.

In the present invention, a means for transferring a desired gene is not particularly limited, and a suitable means selected from known gene introduction methods can be used. As the gene transfer method, either a method using a virus vector or a method not using the vector can be used in the present invention. With respect to details of these methods, many literatures have been already published.

The virus vector may be not particularly limited, and a known virus vector which is usually used in a gene transfer method, for example, a retrovirus vector (including a lentivirus vector and a pseudotyped vector), an adenovirus vector, an adeno-associated virus vector, a simian virus vector, a vaccinia virus vector, a sendaivirus vector or the like can be used. Particularly preferably, a retrovirus vector, an adenovirus vector or a lentivirus vector is used. As the virus vector, preferred is a virus vector lacking the replication ability so as not to self-replicate in an infected cell. For example, the viral vector may comprise a vector comprising a T-cell receptor gene.

Examples of the gene transfer method not using a virus vector which can be used in the present invention include, but not limited to, a method using a carrier such as liposome or ligand-polylysine, a calcium phosphate method, an electroporation method, and a particle gun method. In this case, a foreign gene incorporated into a plasmid DNA or a straight DNA or RNA is transferred.

A retrovirus vector and a lentivirus vector can stably integrate a foreign gene inserted in the vector into the chromosomal DNA of a cell into which the vector is transferred, and they are used for the purpose of gene therapy or the like. Since the vectors can infect cells undergoing division or growth, they are particularly preferably used for performing gene transfer in the process of the present invention.

For example, a desired gene can be inserted into a vector, a plasmid or the like so as to express the gene under the control of a suitable promoter. In addition, in order to attain efficient transcription of the gene, another regulatory element that cooperates with a promoter or a transcription initiation site, for example, an enhancer sequence or a terminator sequence may be present in the vector. In addition, for the purpose of insertion by homologous recombination of the desired gene into the chromosome of a target T cell, for example, the gene may be placed between flanking sequences comprising nucleotide sequences, each having homology with nucleotide sequences present on the both sides of a desired target insertion site of the gene in the chromosome.

Examples of the vector that can be used in the present invention include retrovirus vectors such as a MFG vector, an α-SGC vector (WO 92/07943), pBabe [Morgenstern J. P., Land H., Nucleic Acids Research, vol. 18, No. 12, pp. 3587-3596 (1990)], pLXIN (manufactured by Clontech), and pDON-AI (manufactured by TAKARA BIO INC.), lentivirus vectors [a human immunodeficiency virus (HIV)-derived vector and a simian immunodeficiency virus (SIV)-derived vector], and vectors obtained by modifying them.

In addition, these vectors can be prepared as virus particles in which the vectors are packaged, by using known packaging cell lines, for example, PG13 (ATCC CRL-10686), PG13/LNc8 (ATCC CRL-10685), PA317 (ATCC CRL-9078), GP+E-86 (ATCC CRL-9642), GP+envAm12 (ATCC CRL-9641), and ψCRIP described in Proceedings of the National Academy of Sciences of the USA, vol. 85, pp. 6460-6464 (1988). In addition, retrovirus-producer cells can be also prepared using a 293 cell or a 293 T cell having a high transfection efficiency.

In the present disclosure, a retrovirus prepared by pseudotyped packaging which has an envelope derived from a different virus from a virus from which the genome of the retrovirus is derived, can be also used. For example, a pseudotyped retrovirus having an envelope derived from a molony mouse leukemia virus (MoMLV), a gibbon ape leukemia virus (GaLV), a vesicular stomatitis virus (VSV) or a feline endogenous virus, or a protein capable of functioning as an envelope can be used. Further, a retrovirus having, on a surface thereof, a sugar chain-modified protein prepared by using a retrovirus-producer cell into which a gene of an enzyme involved in sugar chain synthesis or the like is transferred can be used also in the present invention. The above-mentioned virus can be prepared using a packaging cell expressing each envelope. As the packaging cell, a variety of packaging cells have been already reported, and some of them are commercially available. In the present invention, these known packaging cells can be used.

When gene transfer is carried out using a retrovirus vector, a functional substance having retrovirus-binding activity can be used to improve a gene transfer efficiency. Examples of the functional substance having retrovirus-binding activity used in the process include, but not particularly limited to, a heparin-II-binding region of fibronectin, a fibroblast growth factor, V-type collagen, fragments of the above-mentioned polypeptides, polylysine, and DEAE-dextran. It is preferable that the fibronectin fragment has a heparin-II-binding region in the molecule, and such a fragment is also described in WO 95/26200 and WO 97/18318. CH-296, which is a fibronectin fragment having a heparin-II-binding region, is commercially available under the name of RetroNectin® (recombinant human fibronectin fragment). In addition, substances functionally equal to these functional substances, for example, a functional substance having a heparin-binding site can be also used. In addition, a mixture of the functional substances, a polypeptide containing the functional substance, a polymer of the functional substance, a derivative of the functional substance and the like can be also used.

In addition, a functional substance having target cell-binding activity may be used together in the present invention. The substance is useful for improving a gene transfer efficiency into a target cell or performing target cell-specific gene transfer. An example of the functional substance having target cell-binding activity is, but not particularly limited to, a substance having a ligand capable of binding to a target cell. Examples of the ligand include a cell-adherent protein (fibronectin, laminine, collagen and the like) or a fragment thereof, a hormone, a cytokine, an antibody to an antigen on the cell surface, a polysaccharide, a glycoprotein, a glycolipid, a sugar chain derived from a glycoprotein or a glycolipid, and metabolites of a target cell. In addition, a polypeptide containing the functional substance, a polymer of the functional substance, a derivative of the functional substance, and a functionally equivalent substance of the functional substance can also be used. The functional substance having target cell-binding activity may be immobilized onto a solid phase, like the functional substance having retrovirus-binding activity. As the functional substance having retrovirus-binding activity, a substance having target cell-binding activity in addition to retrovirus-binding activity can be also used.

As explained above, according to the process of the present disclosure including gene transfer after culture in the presence of the active ingredients containing retinoic acid, it becomes possible to efficiently perform gene transfer into a cell population containing a T cell. In addition, the process of the present disclosure may not require a special facility or apparatus. Many kinds of retrovirus vectors and target cells may be effective in the process of the present disclosure. Further, since the process of the present disclosure may be suitable for utilization in a closed system, it may be very clinically useful for gene therapy and the like.

An efficiency of transferring a gene into a T cell is improved by carrying out a gene transfer operation after the step of activating in the presence of RA. That is, the cell population prepared by the process of the present disclosure may be a cell population containing a high proportion of a T cell into which a desired gene, e.g., TCR, is transferred. The methods described herein may be particularly useful in preparation of cells for gene therapy.

### Patient Population

The disclosure provides methods of treating a patient or individual having a cancer or in need of a treatment thereof, comprising administering to the patient an effective amount of the expanded and transduced T cells described herein.

The patient or individual in need thereof may be a cancer patient. The cancer to be treated by the methods and compositions described herein may be hepatocellular carcinoma (HCC), colorectal carcinoma (CRC), glioblastoma (GB), gastric cancer (GC), esophageal cancer, non-small cell lung cancer (NSCLC), pancreatic cancer (PC), renal cell carcinoma (RCC), benign prostate hyperplasia (BPH), prostate cancer (PCA), ovarian cancer (OC), melanoma, breast cancer, chronic lymphocytic leukemia (CLL), Merkel cell carcinoma (MCC), small cell lung cancer (SCLC), Non-Hodgkin lymphoma (NHL), acute myeloid leukemia (AML), gallbladder cancer and cholangiocarcinoma (GBC, CCC), urinary bladder cancer (UBC), acute lymphocytic leukemia (ALL), uterine cancer (UEC), or a combination thereof where the patient suffers from more than one type of cancer.

### Pharmaceutical Compositions

To facilitate administration, the transduced T cells according to the disclosure can be made into a pharmaceutical composition or made into an implant appropriate for administration in vivo, with appropriate carriers or diluents, which further can be pharmaceutically acceptable. The means of making such a composition or an implant have been described in the art (See, for instance, Remington's Pharmaceutical Sciences, 16th Ed., Mack, ed. (1980)). Where appropriate, the transduced T cells can be formulated into a preparation in semisolid or liquid form, such as a capsule, solution, injection, inhalant, or aerosol, in the usual ways for their respective route of administration. Means known in the art can be utilized to prevent or minimize release and absorption of the composition until it reaches the target tissue or organ, or to ensure timed-release of the composition. Desirably, however, a pharmaceutically acceptable form is employed that does not ineffectuate the cells expressing the TCR. Thus, desirably the transduced T cells can be made into a pharmaceutical composition containing a balanced salt solution, preferably Hanks' balanced salt solution, or normal saline.

A composition of the present invention can be provided in unit dosage form wherein each dosage unit, e.g., an injection, contains a predetermined amount of the composition, alone or in appropriate combination with other active agents. The specifications for the novel unit dosage forms of the present invention depend on the particular pharmacodynamics associated with the pharmaceutical composition in the particular subject.

Compositions may comprise an effective amount of the isolated transduced T cells and be introduced into the subject such that long-term, specific, anti-tumor responses is achieved to reduce the size of a tumor or eliminate tumor growth or regrowth than would otherwise result in the absence of such treatment. For example, the amount of transduced T cells reintroduced into the subject causes an about 10%, about 20%, about 30%, about 40%, about 50%, about 60%, about 70%, about 80%, about 90%, about 95%, about 98%, or about 99% decrease in tumor size when compared to otherwise same conditions where the transduced T cells are not present.

Accordingly, the amount of transduced T cells administered may take into account the route of administration and should be such that a sufficient number of the transduced T cells will be introduced so as to achieve the desired therapeutic response. Furthermore, the amounts of each active agent included in the compositions described herein (e.g., the amount per each cell to be contacted or the amount per certain body weight) can vary in different applications. In general, the concentration of transduced T cells desirably should be sufficient to provide in the subject being treated, for example, effective amounts of transduced T cells may be about 1×10⁶ to about 1×10⁹ transduced T cells/m² (or kg) of a patient, even more desirably, from about 1×10⁷ to about 5×10⁸ transduced T cells/m² (or kg) of a patient. Any suitable amount can be utilized, e.g., greater than 5×10⁸ cells/m² (or kg) of a patient, or below, e.g., less than 1×10⁷ cells/m² (or kg) of a patient, as is necessary to achieve a therapeutic effect. The dosing schedule can be based on well-established cell-based therapies (See, e.g., U.S. Pat. No. 4,690,915), or an alternate continuous infusion strategy can be employed.

In an aspect, T cells or a population of T cells described herein selectively recognize cancer cells that present a peptide. In an aspect, T cells or a population of described herein selectively recognize cancer cells that present a peptide in, for example, those peptides described in U.S. Patent Application Publication Nos. 2016/0187351; 2017/0165335; 2017/0035807; 2016/0280759; 2016/0287687; 2016/0346371; 2016/0368965; 2017/0022251; 2017/0002055; 2017/0029486; 2017/0037089; 2017/0136108; 2017/0101473; 2017/0096461; 2017/0165337; 2017/0189505; 2017/0173132; 2017/0296640; 2017/0253633; 2017/0260249; 2018/0051080, and 2018/0164315. In an aspect, T cells described herein selectively recognize cells which present a TAA peptide described in one of more of the patents and publications described herein. In an aspect, T cells described herein selectively recognize cells which present a tumor associated antigen (TAA) peptide described in one of more of the patents and publications described above.

In another aspect, TAA that are capable of use with the methods and embodiments described herein include at least one selected from SEQ ID NO: 1 to SEQ ID NO: 157. In an aspect, T cells selectively recognize cells which present a TAA peptide described in SEQ ID NO: 1-157 or any of the patents or applications described herein.

| SEQ ID | Amino Acid |
|---|---|
| NO: | Sequence |
| 1 | YLYDSETKNA |
| 2 | HLMDQPLSV |
| 3 | GLLKKINSV |
| 4 | FLVDGSSAL |
| 5 | FLFDGSANLV |
| 6 | FLYKIIDEL |
| 7 | FILDSAETTTL |
| 8 | SVDVSPPKV |
| 9 | VADKIHSV |
| 10 | IVDDLTINL |
| 11 | GLLEELVTV |
| 12 | TLDGAAVNQV |
| 13 | SVLEKEIYSI |
| 14 | LLDPKTIFL |
| 15 | YTFSGDVQL |
| 16 | YLMDDFSSL |
| 17 | KVWSDVTPL |
| 18 | LLWGHPRVALA |
| 19 | KIWEELSVLEV |
| 20 | LLIPFTIFM |
| 21 | FLIENLLAA |
| 22 | LLWGHPRVALA |
| 23 | FLLEREQLL |
| 24 | SLAETIFIV |
| 25 | TLLEGISRA |
| 26 | ILQDGQFLV |
| 27 | VIFEGEPMYL |
| 28 | SLFESLEYL |
| 29 | SLLNQPKAV |
| 30 | GLAEFQENV |
| 31 | KLLAVIHEL |
| 32 | TLHDQVHLL |
| 33 | TLYNPERTITV |
| 34 | KLQEKIQEL |
| 35 | SVLEKEIYSI |
| 36 | RVIDDSLVVGV |
| 37 | VLFGELPAL |
| 38 | GLVDIMVHL |
| 39 | FLNAIETAL |
| 40 | ALLQALMEL |
| 41 | ALSSSQAEV |
| 42 | SLITGQDLLSV |
| 43 | QLIEKNWLL |
| 44 | LLDPKTIFL |
| 45 | RLHDENILL |
| 46 | YTFSGDVQL |
| 47 | GLPSATTTV |
| 48 | GLLPSAESIKL |
| 49 | KTASINQNV |
| 50 | SLLQHLIGL |
| 51 | YLMDDFSSL |
| 52 | LMYPYIYHV |
| 53 | KVWSDVTPL |
| 54 | LLWGHPRVALA |
| 55 | VLDGKVAVV |
| 56 | GLLGKVTSV |
| 57 | KMISAIPTL |
| 58 | GLLETTGLLAT |
| 59 | TLNTLDINL |
| 60 | VIIKGLEEI |
| 61 | YLEDGFAYV |
| 62 | KIWEELSVLEV |
| 63 | LLIPFTIFM |
| 64 | ISLDEVAVSL |
| 65 | KISDFGLATV |
| 66 | KLIGNIHGNEV |
| 67 | ILLSVLHQL |
| 68 | LDSEALLTL |
| 69 | VLQENSSDYQSNL |
| 70 | HLLGEGAFAQV |
| 71 | SLVENIHVL |
| 72 | YTFSGDVQL |
| 73 | SLSEKSPEV |
| 74 | AMFPDTIPRV |
| 75 | FLIENLLAA |
| 76 | FTAEFLEKV |
| 77 | ALYGNVQQV |
| 78 | LFQSRIAGV |
| 79 | ILAEEPIYIRV |
| 80 | FLLEREQLL |
| 81 | LLLPLELSLA |
| 82 | SLAETIFIV |
| 83 | AILNVDEKNQV |
| 84 | RLFEEVLGV |
| 85 | YLDEVAFML |
| 86 | KLIDEDEPLFL |
| 87 | KLFEKSTGL |
| 88 | SLLEVNEASSV |
| 89 | GVYDGREHTV |
| 90 | GLYPVTLVGV |
| 91 | ALLSSVAEA |
| 92 | TLLEGISRA |
| 93 | SLIEESEEL |
| 94 | ALYVQAPTV |
| 95 | KLIYKDLVSV |
| 96 | ILQDGQFLV |
| 97 | SLLDYEVSI |
| 98 | LLGDSSFFL |
| 99 | VIFEGEPMYL |
| 100 | ALSYILPYL |
| 101 | FLFVDPELV |
| 102 | SEWGSPHAAVP |
| 103 | ALSELERVL |
| 104 | SLFESLEYL |
| 105 | KVLEYVIKV |
| 106 | VLLNEILEQV |
| 107 | SLLNQPKAV |
| 108 | KMSELQTYV |
| 109 | ALLEQTGDMSL |
| 110 | VIIKGLEEITV |
| 111 | KQFEGTVEI |
| 112 | KLQEEIPVL |
| 113 | GLAEFQENV |
| 114 | NVAEIVIHI |
| 115 | ALAGIVTNV |
| 116 | NLLIDDKGTIKL |
| 117 | VLMQDSRLYL |
| 118 | KVLEHVVRV |
| 119 | LLWGNLPEI |
| 120 | SLMEKNQSL |
| 121 | KLLAVIHEL |
| 122 | ALGDKFLLRV |
| 123 | FLMKNSDLYGA |
| 124 | KLIDHQGLYL |
| 125 | GPGIFPPPPPQP |
| 126 | ALNESLVEC |
| 127 | GLAALAVHL |
| 128 | LLLEAVWHL |
| 129 | SIIEYLPTL |
| 130 | TLHDQVHLL |
| 131 | SLLMWITQC |
| 132 | FLLDKPQDLSI |
| 133 | YLLDMPLWYL |
| 134 | GLLDCPIFL |
| 135 | VLIEYNFSI |
| 136 | TLYNPERTITV |
| 137 | AVPPPPSSV |
| 138 | KLQEELNKV |
| 139 | KLMDPGSLPPL |
| 140 | ALIVSLPYL |
| 141 | FLLDGSANV |
| 142 | ALDPSGNQLI |
| 143 | ILIKHLVKV |
| 144 | VLLDTILQL |
| 145 | HLIAEIHTA |
| 146 | SMNGGVFAV |
| 147 | MLAEKLLQA |
| 148 | YMLDIFHEV |
| 149 | ALWLPTDSATV |
| 150 | GLASRILDA |
| 151 | SYVKVLHHL |
| 152 | VYLPKIPSW |
| 153 | NYEDHFPLL |
| 154 | VYIAELEKI |
| 155 | VHFEDTGKTLLF |
| 156 | VLSPFILTL |
| 157 | HLLEGSVGV |

Although the invention has been described in some detail by way of illustration and example for purposes of clarity of understanding, it should be understood that certain changes and modifications may be practiced within the scope of the appended claims. Modifications of the above-described modes for carrying out the invention that would be understood in view of the foregoing disclosure or made apparent with routine practice or implementation of the invention to persons of skill in oncology, physiology, immunology, and/or related fields are intended to be within the scope of the following claims.

All publications (e.g., Non-Patent Literature), patents, patent application publications, and patent applications mentioned in this specification are indicative of the level of skill of those skilled in the art to which this invention pertains. All such publications (e.g., Non-Patent Literature), patents, patent application publications, and patent applications are herein incorporated by reference to the same extent as if each individual publication, patent, patent application publication, or patent application was specifically and individually indicated to be incorporated by reference.

### Example 1

### Experimental Procedures

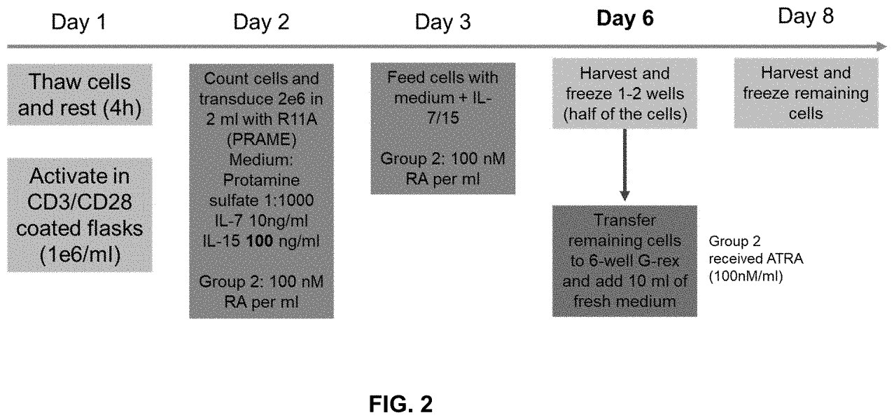

FIG. 2 shows general experimental timeline. Briefly, on Day 1, cryopreserved Peripheral blood mononuclear cells (PBMCs) obtained from leukapheresis products were thawed and rested for about 4 hours, followed by overnight activation in flasks at a density of about 1×10⁶ cells/ml without all trans retinoic acid (RA) (Group 1) or with all-trans-retinoic acid (ATRA) (100 nM/ml) (Group 2), flasks were previously coated with anti-CD3 antibody and anti-CD28 antibody. On Day 2, cells were counted and 2×10⁶ cells in 2 ml were transduced by viral vector, e.g., lentiviral vector, expressing a TCR, e.g., a TCR binding to a PRAME peptide/MHC complex in media containing protamine sulfate (1:1000), IL-7 (10 ng/ml), and IL-15 (100 ng/ml) without all trans retinoic acid (RA) (Group 1) or with ATRA (100 nM/ml) (Group 2). On Day 3, cells were fed with media containing IL-7 and IL-15. On Day 6, cells were harvested and about half of cells were cryopreserved. The other half of cells were transferred to G-Rex 6-well plate, to which 10 ml of fresh medium was added without RA (Group 1) or with RA (100 nM/ml) (Group 2). Some Day 6 cells were analysed by flow cytometry to assess transduction efficiency. On Day 8, remaining cells were harvested and cryopreserved.

### RA Reduces the Number of Dextramer Specific Cells

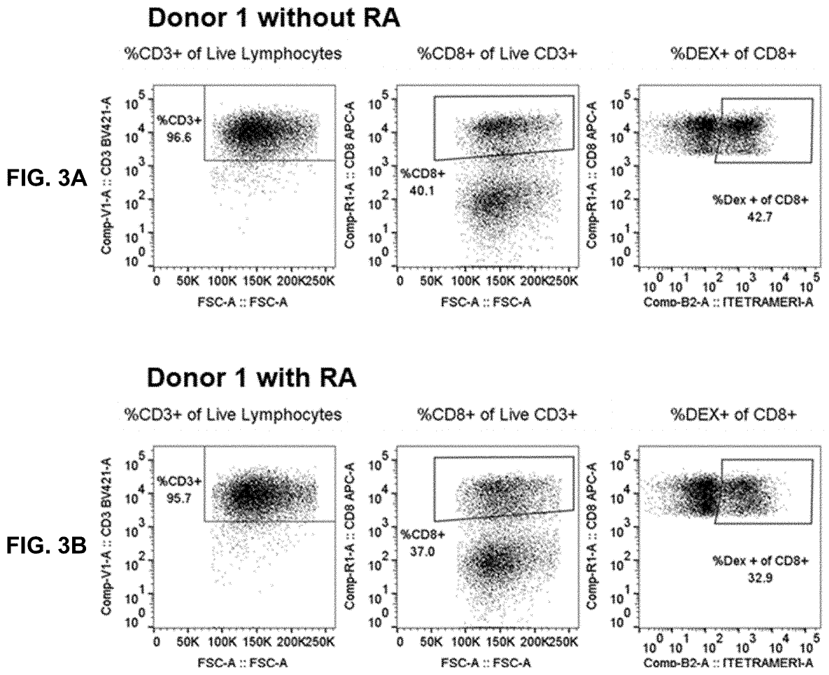

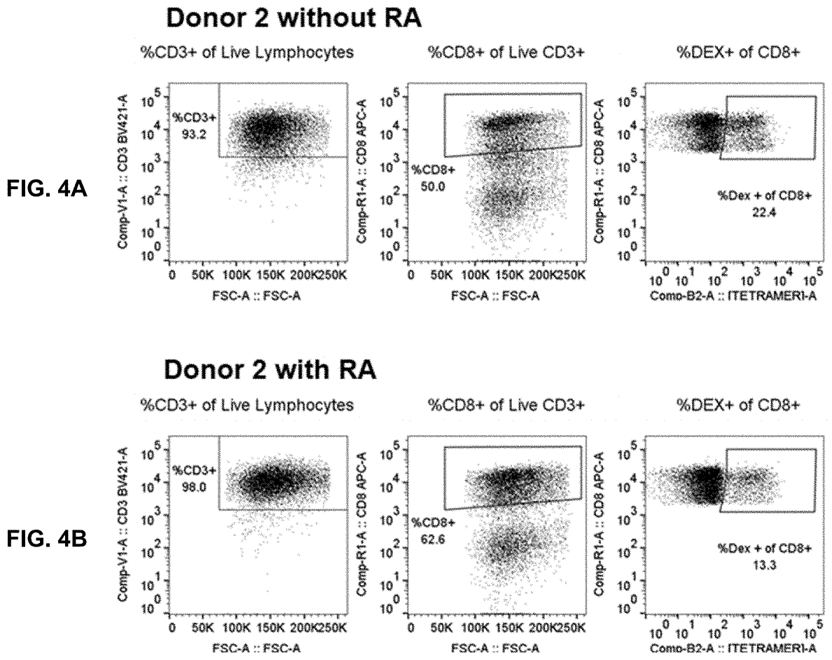

Flow cytometry staining was performed on Day 6 cells to assess the expression of TCR. FIG. 3A (right panel) shows, using T cells obtained from Donor 1, 42.7% of CD8+ cells expressing the transduced TCR without RA treatment as compared with 32.9% of CD8+ cells expressing the transduced TCR with RA treatment (FIG. 3B (right panel)), while % CD3+ of live lymphocytes (FIG. 3A (left panel) and FIG. 3B (left panel)) and % CD8+ of live CD3+ cell (FIG. 3A (middle panel) and FIG. 3B (middle panel)) appear to be comparable. Similarly, FIG. 4A (right panel) shows, using T cells obtained from Donor 2, 22.4% of CD8+ cells expressing the transduced TCR without RA treatment as compared with 13.3% of CD8+ cells expressing the transduced TCR with RA treatment (FIG. 4B (right panel)), while % CD3+ of live lymphocytes (FIG. 4A (left panel) and FIG. 4B (left panel)) and % CD8+ of live CD3+ cell (FIG. 4A (middle panel) and FIG. 4B (middle panel)) appear to be comparable.

Table 1 shows flow cytometry panels for T cell products manufactured in the presence of RA.

| Fluoro- | | | | | Metabolic/ | | NK related | |
|---|---|---|---|---|---|---|---|---|
| chrome | Tmem | Dilution | Homing | Dilution | activation | Dilution | markers | Dilution |
| AX488 | CD8 | 1:80 | CD8 | 1:80 | CD8 | 1:80 | CD94 | 1:80 |
| PerCP- | CD3 | 1:300 | CD3 | 1:300 | CD3 | 1:300 | CD3 | 1:300 |
| Cy5.5 | | | | | | | | |
| PE | Prame | 1:30 | Prame | 1:30 | Prame | 1:30 | Prame | 1:30 |
| | DEXTRAMER | | DEXTRAMER | | DEXTRAMER | | DEXTRAMER | |
| Pe- | CD27 | 1:80 | CCR9 | 1:80 | CD38 | 1:80 | KLRG1 | 1:80 |
| dazzle594 | | | | | | | | |
| PE-Cy7 | CD95 | 1:80 | CD49a | 1:80 | CD69 | 1:80 | CD159a | 1:40 |
| | | | | | | | (NKG2A) | |
| APC | CD25 | 1:80 | CD103 | 1:80 | CD73 | 1:80 | CD57 | 1:80 |
| AX700 | ICOS | 1:80 | Beta 7 | 1:120 | empty | | CD161 | 1:80 |
| APC- | CD45RA | 1:300 | CD45RA | 1:300 | CD45RA | 1:300 | CD45RA | 1:300 |
| fire750 | | | | | | | | |
| BV421 | CCR7 | 1:40 | CXCR4 | 1:80 | CCR7 | 1:40 | CCR7 | 1:40 |
| Aqua | Live/dead | 1:1000 | Live/dead | 1:1000 | Live/dead | 1:1000 | Live/dead | 1:1000 |
| | BV510 | | BV510 | | BV510 | | BV510 | |
| BV605 | CD45RO | 1:80 | CD45RO | 1:80 | CD45RO | 1:80 | CD8 | 1:100 |
| BV650 | CD28 | 1:80 | empty | | CD39 | 1:80 | CD56 | 1:80 |
| BV711 | Ki-67 | 1:60 | CD49d | 1:80 | CD95 | 1:80 | empty | |
| | (ICS) | | | | | | | |
| BV785 | CD62L | 1:200 | CCR6 | 1:80 | HLA-DR | 1:80 | CD314 | 1:80 |
| | | | | | | | (NKG2D) | |

### RA Increases CD45RO+ T Cells

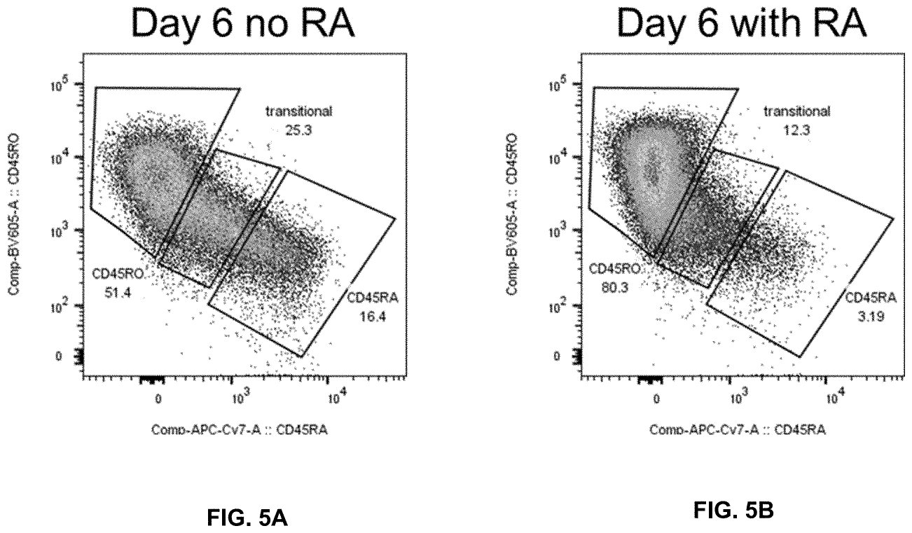

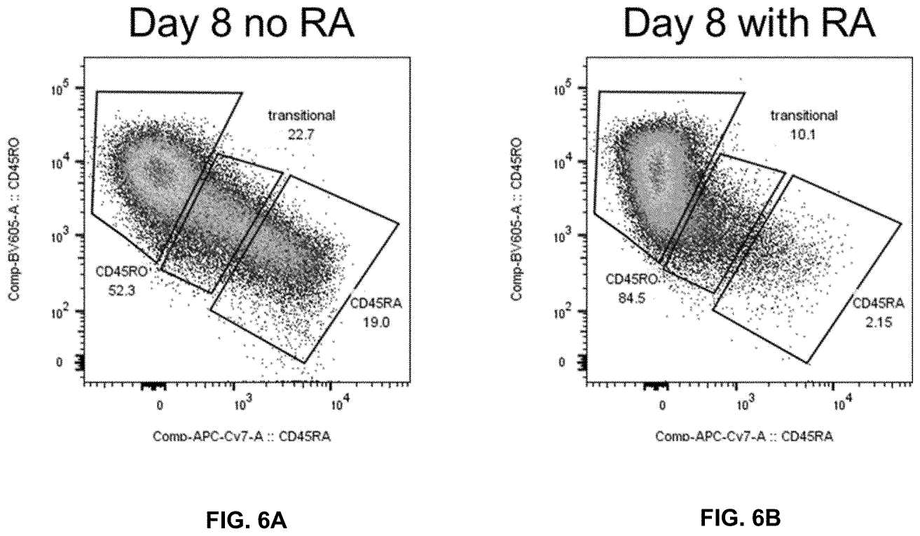

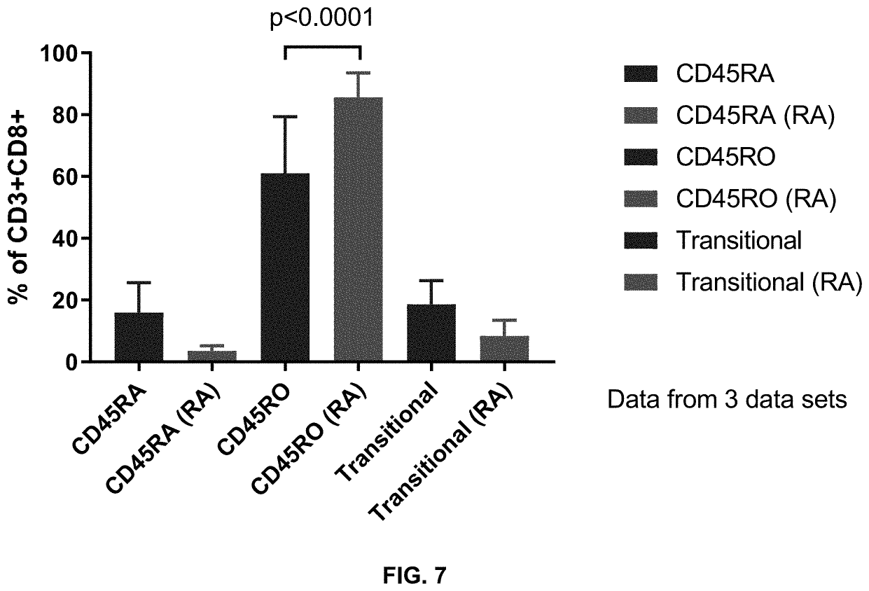

FIG. 5B shows RA-treated Day 6 T cell products have more CD45RO+ T cells (80.3%) than that without RA treatment (FIG. 5A, 51.4%). Similarly, FIG. 6B shows RA-treated Day 8 T cell products have more CD45RO+ T cells (84.5%) than that without RA treatment (FIG. 6A, 52.3%). FIG. 7 shows, using 1-way ANOVA on 3 data sets, RA treatment significantly increases CD45RO+ T cells and significantly decreases both CD45RA+ and transitional CD3+CD8+ T cells.

RA Decreases naïve/Tscm (CD45RA+CD197+)

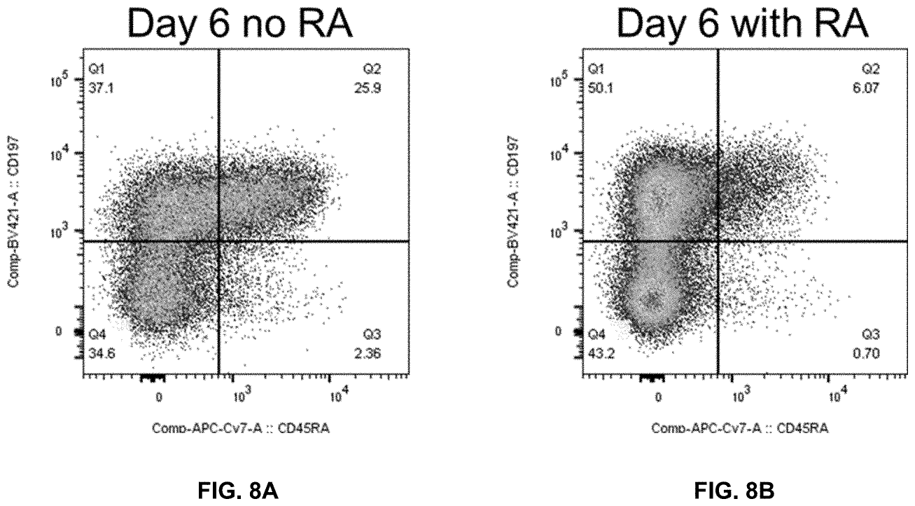

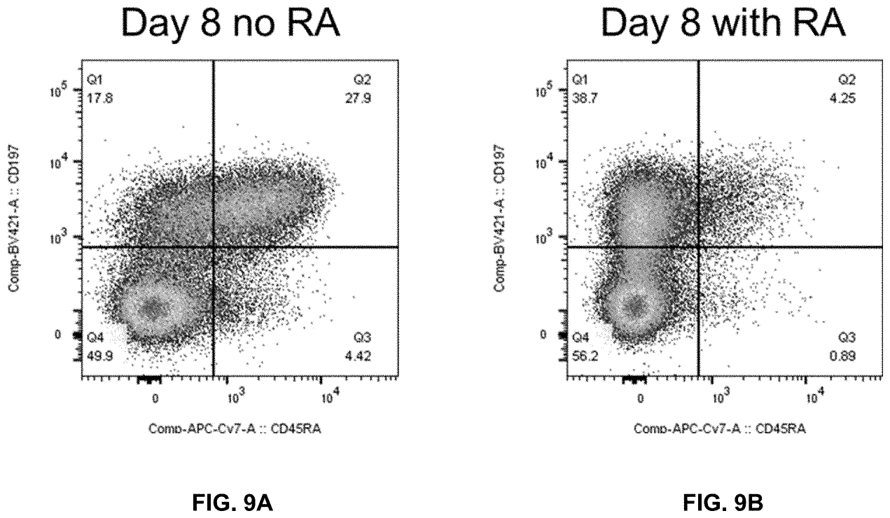

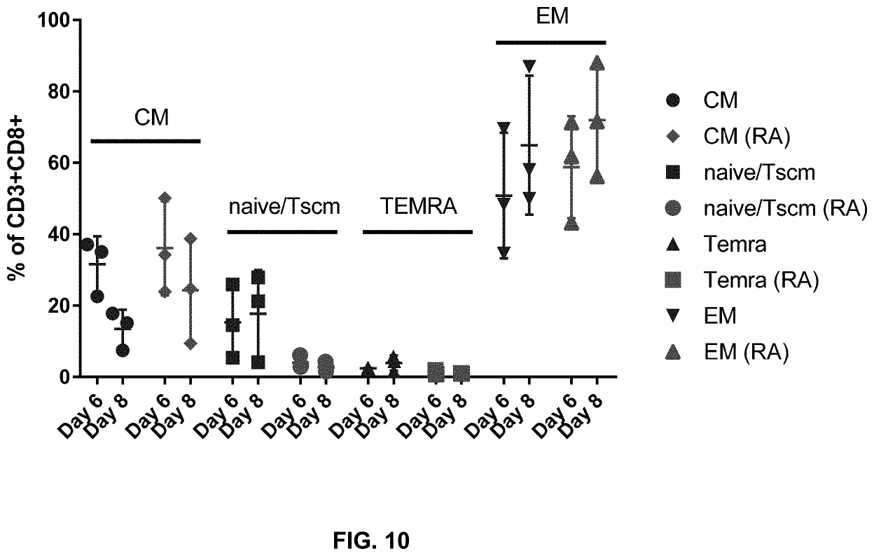

FIG. 8B shows RA-treated Day 6 T cell products have fewer naïve/Tscm (CD45RA+CD197+) (6.07%) than that without RA treatment (FIG. 8A, 25.9%). Similarly, FIG. 9B shows RA-treated Day 8 T cell products have fewer naïve/T_{scm} (CD45RA+CD197+) (4.25%) than that without RA treatment (FIG. 9A, 27.9%). FIG. 10 shows RA treatment significantly decreases naïve/Tscm and has little effect on Tₑₘᵣₐ and Tₑₘ. Consistent to FIGS. 8A-9B, FIG. 10 shows RA treatment increases T_{cm}.

### RA Decreases CD62L+ T Cells

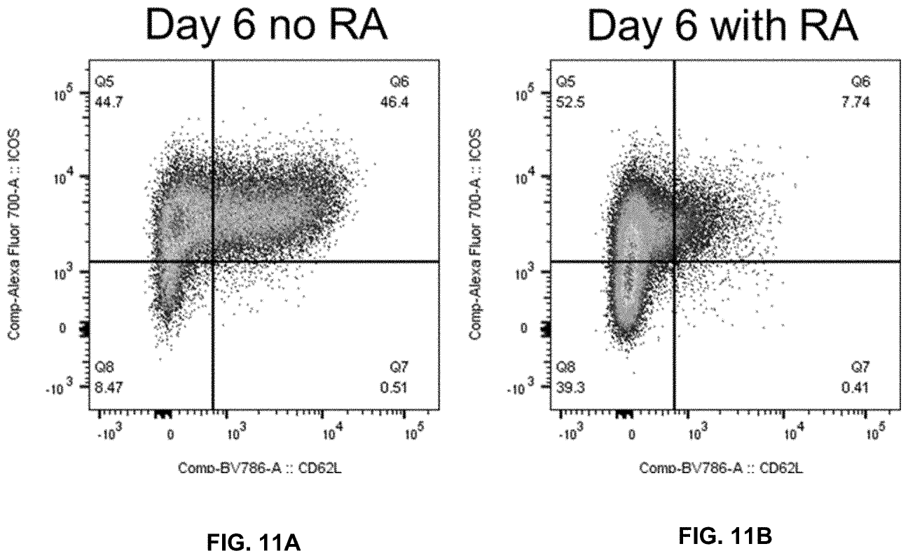

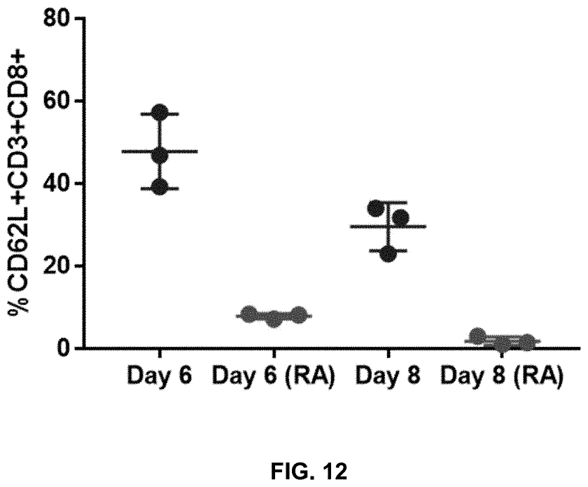

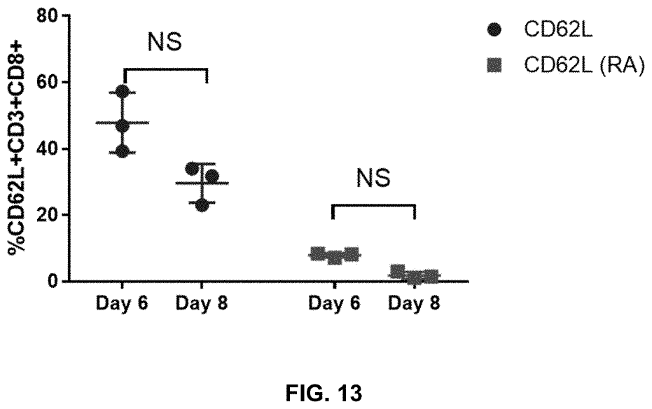

FIG. 11B shows RA-treated Day 6 T cell products have fewer CD62L+ T cells (7.74%) than that without RA treatment (FIG. 11A, 46.4%). Similarly, FIG. 12 shows RA-treated Day 8 T cell products have significantly fewer CD62L+ T cells than that without RA treatment. FIG. 13 shows no significant difference (Wilcoxon test) in CD62L+ T cells between Day 6 and Day 7 T cell products regardless RA treatment.

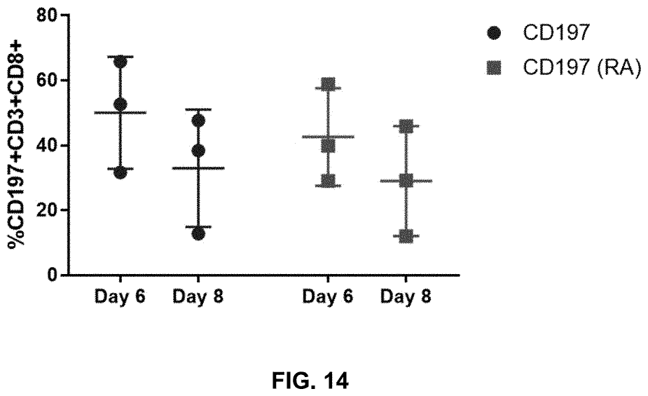

FIG. 14 shows no significant difference in CD197+ cells between Day 6 and Day 8 T cell products with or without RA treatment.

RA does not impact the expression of CD25, which is downregulated with longer cell culture

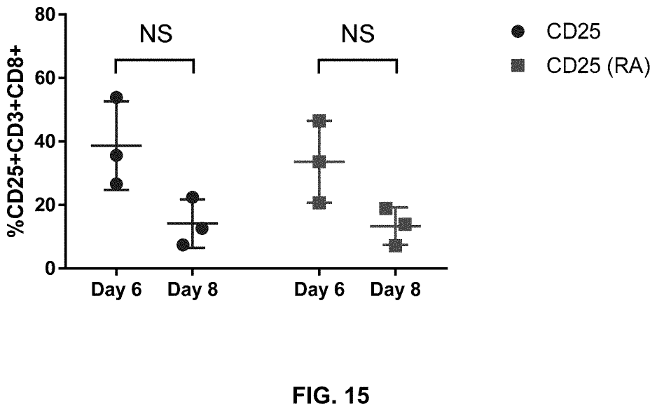

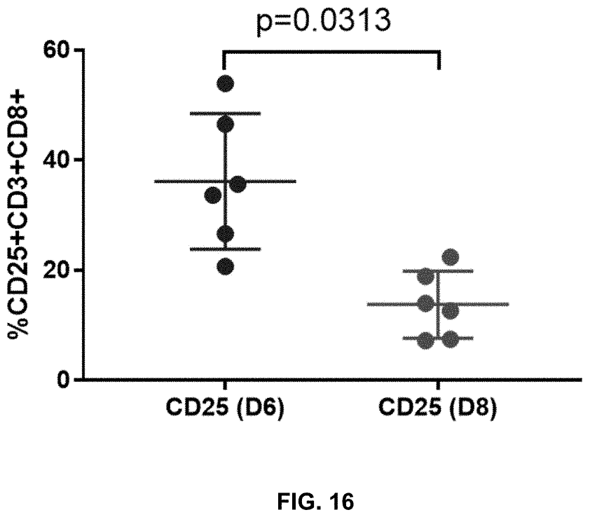

FIG. 15 shows no significant difference (Paired t-test) in CD25+ cells between that with and without RA treatment in Day 6 or Day 8 T cell products, although Day 8 RA-treated T cell products appear to have significantly fewer CD25+ cells than Day 6 T cell products without RA treatment, as shown in FIG. 16 (Wilcoxon test, p=0.0313).

RA-treated cells express higher levels of Ki-67 but lower levels of CD28 family member Inducible T-cell COStimulator (ICOS) (or CD278).

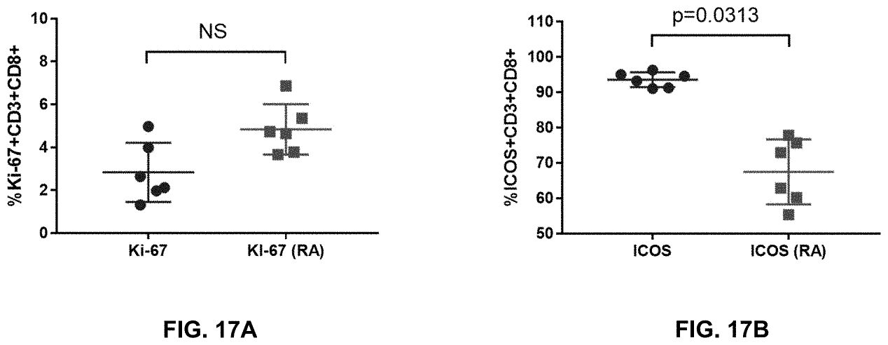

ICOS is a CD28-superfamily costimulatory cell surface receptor molecule that is expressed on activated T cells. ICOS forms homodimers and plays an important role in cell-cell signaling, immune responses and regulation of cell proliferation, e.g., for Th2 cells. FIG. 17A shows no significant difference (Wilcoxon test) in Ki-67+ cells between T cell products with or without RA treatment, suggesting RA treatment may not affect cell proliferation. ICOS is expressed on activated T cells. FIG. 17B shows RA treatment significantly decreases ICOS+ cells (Wilcoxon test, p=0.0313), suggesting RA treatment may decrease activated T cells. There is, however, no difference in Ki-67+ cells and ICOS+ cells between Day 6 and Day 8 T cell products.

### RA Increases CD38+ T Cells

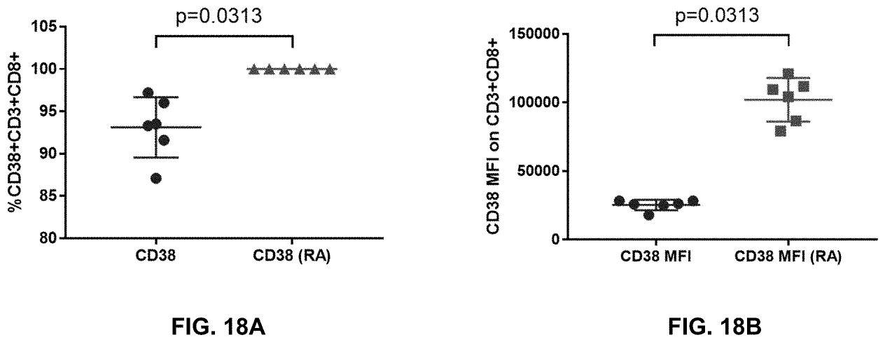

Memory T lymphocytes are CD38-positive. FIGS. 18A and 18B show RA treatment significantly increases CD38+ cells (Wilcoxon test, p=0.0313), suggesting RA treatment may increase memory T cells. CD38 is expressed on all RA-treated CD8+ T cells. Similarly, CD38 MFI (Mean Fluorescence Intensity) is higher in RA-treated CD8+ T cells than that without RA treatment (Wilcoxon test, p=0.0313). There is, however, no difference in Ki-67+ cells and CD38 MFI between Day 6 and Day 8 T cell products.

### RA Upregulates CD69 but not CD103

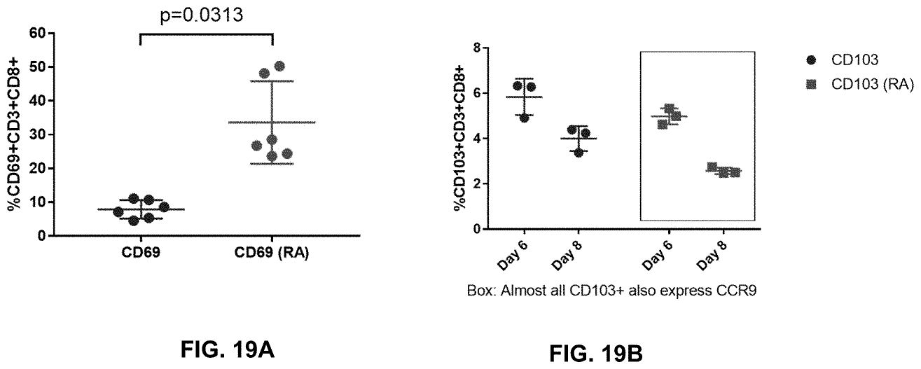

CD69, CD103 and CD49a are considered tissue resident memory (Trm) markers. FIG. 19A shows RA treatment significantly increases CD69+ cells (Wilcoxon test, p=0.0313). There is, however, no difference in CD69+ cells between Day 6 and Day 8 T cell products with or without RA treatment. These results suggest that these T cell products may be Trm. In contrast, FIG. 19B shows RA treatment does not significantly affect CD103 expression, although Day 8 T cell products appear to have fewer CD103+ cells than Day 6 T cell products. In addition, almost all RA-treated CD103+ cells also express CCR9.

### RA Upregulates Mucosal Homing Markers CCR9 and α4β7

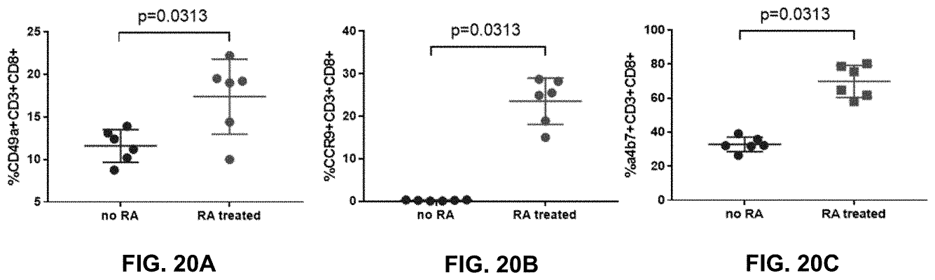

FIGS. 20A, 20B, and 20C show RA treatment significantly increases CD49a+ cells, CCR9+ cells, and α4β7+ cells, respectively (Wilcoxon test, p=0.0313). α4β7+ cells were measured by detecting α4+ cells and β7+ cells separately. There is, however, no difference in CD49a+ cells, CCR9+ cells, and α4β7+ cells between Day 6 and Day 8 T cell products. These results indicate that RA treatment may direct T cells homing to organs, such as intestines, in which T cells expressing CCR9 and α4β7 reside. Thus, RA-treated engineered T cells expressing TCR that binds to tumor associated antigen/MHC complex may be used to treat certain mucosa-related cancers, such as colon cancer.

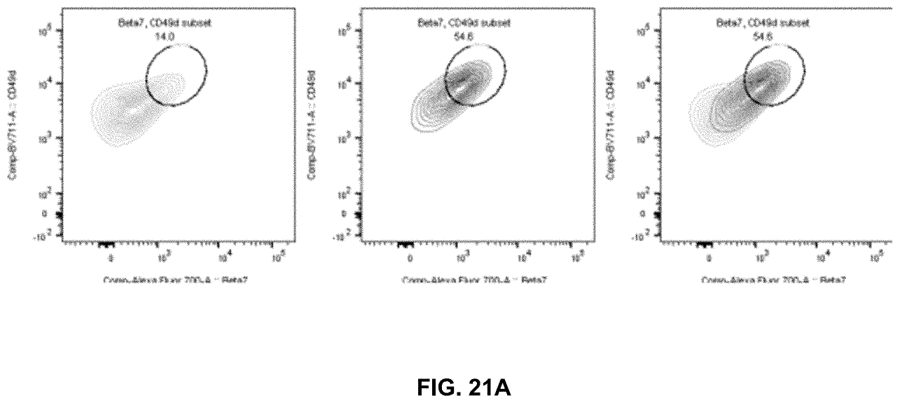

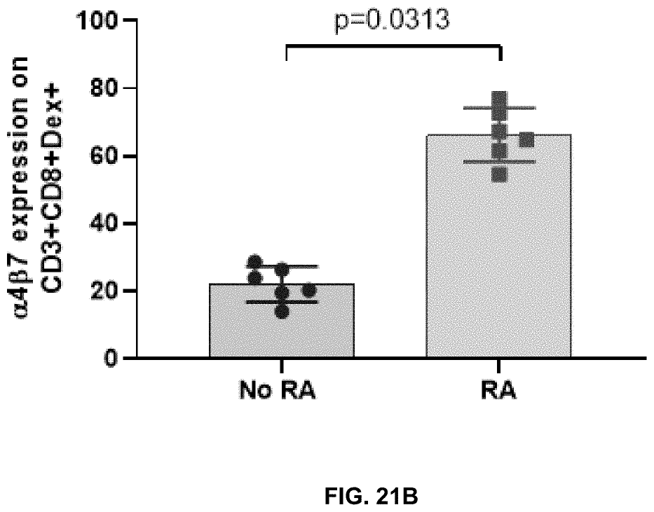

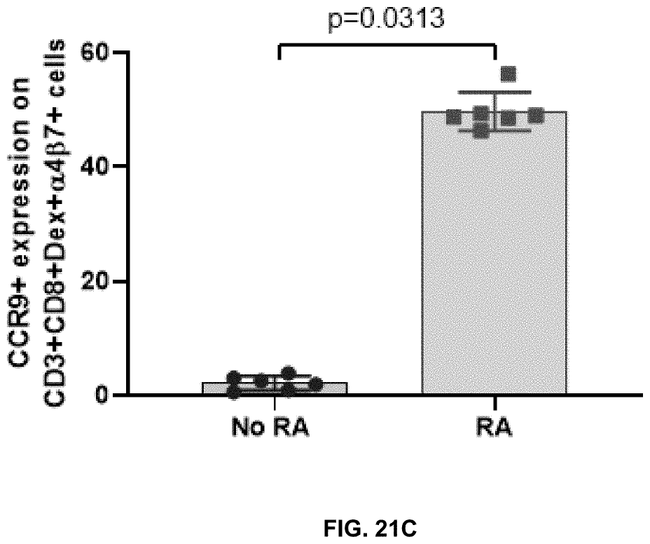

FIG. 21A shows a flow plot of CD49d (α4) and β7 expression on matched CD3+CD8+DEX+ cells, in which, without RA treatment, 14.0% α4β7+ cells were present in CD3+CD8+DEX+ cells (left panel). In contrast, RA treatment resulted in 54.6% α4β7+ cells present in CD3+CD8+DEX+ cells (middle panel). The right panel shows an overlay of that without RA treatment (left panel) versus that with RA treatment (middle panel).

FIG. 21B shows a summary of the data shown in FIG. 21A, e.g., significantly higher α4β7 expression in RA treated CD3+CD8+DEX+ cells than that without RA treatment, p=0.0313. FIG. 21C shows analysis on the cells gated in FIG. 21A for the expression of CCR9, which is a mucosal homing marker. The RA treated group showed significantly higher expression of CCR9 on DEX+CD3+CD8+α4β7+ cells treated with RA than those without RA treatment, p=0.0313.

### RA May not Affect the Amount of CD39+, HLA-DR+, or CXCR4+ Cells

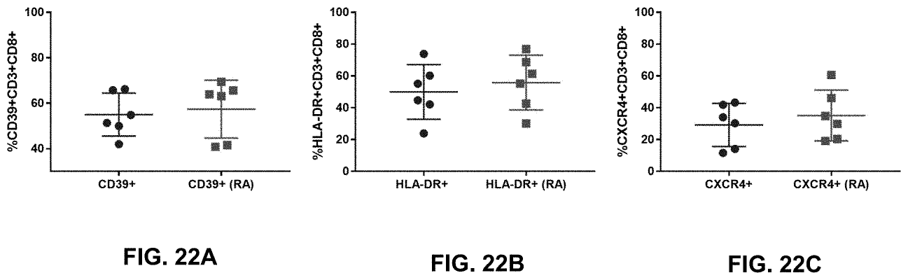

FIGS. 22A, 22B, and 22C show no significant difference in CD39+ cells, HLA-DR+ cells, and CXCR4+ cells, respectively, (Wilcoxon test). In addition, there is no difference in CD39+ cells, HLA-DR+ cells, and CXCR4+ cells, between Day 6 and Day 8 T cell products.

### RA May not Affect the Amount of CD27+ or CD28+ Cells

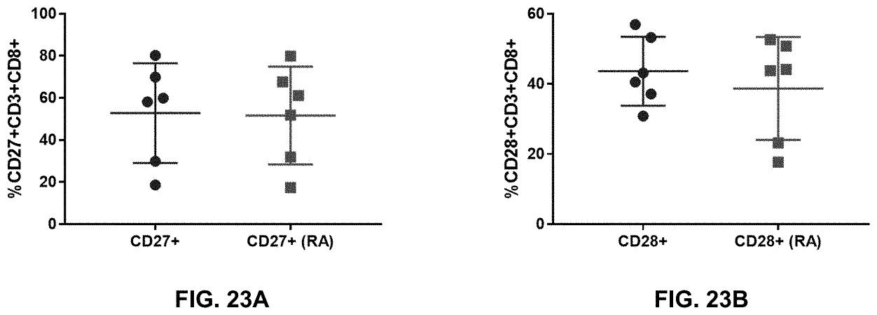

FIGS. 23A and 23B show no significant difference in CD27+ cells and CD28+ cells, respectively, (Wilcoxon test). In addition, there is no difference in CD27+ cells and CD28+ cells between Day 6 and Day 8 T cell products.

Table 2 shows various markers not affected by RA treatment.

| Marker | Description |
|---|---|
| CD39 | no difference was observed between Day 6 or Day 8 cells |
| | untreated or treated with RA |
| CD73 | no difference was observed between Day 6 or Day 8 cells |
| | untreated or treated with RA (<5% positive, data not shown) |
| CD27 | No difference was seen between treated and untreated and |
| CD28 | Day 6 or Day 8 cells. There was a trend to lower values on |
| | longer expanded cells. |
| CD95 | no difference was observed between Day 6 or Day 8 cells |
| | untreated or treated with RA (>90% positive, data not shown) |
| HLA-DR | no difference was observed between Day 6 or Day 8 cells |
| | untreated or treated with RA |
| CXCR4 | no difference was observed between Day 6 or Day 8 cells |
| | untreated or treated with RA |
| CCR6 | to low frequency and no differences (data not shown) |
| CD94 | No difference was seen between treated and untreated and |
| CD159a | Day 6 or Day 8 cells with respect to NK-related markers (data |
| CD314 | not shown). |
| CD161 | |
| CD56 | |
| CD57 | |

To assess the killing activity of T cells treated with or without RA, PRAME TCR (R11A) transduced T cells, e.g., R11A-1, R11A-2, and R11A-3 T cells, were activated and cultured with or without RA, followed by culturing these transduced T cells with low target-positive cells (e.g., A375 having about 51 copies of target peptides per cell) and high target-positive cells (e.g., U2OS having about 242 copies of target peptides per cell) using IncuCyte Killing Assay (E:T ratio 10:1). The assay allows for comparison between RA treated and untreated cells as well as the comparison between Day 6 and Day 8 harvested cells. As controls for the growth curve, target cells alone were cultured. A375 and U2OS were cultured in DMEM, however, the killing assay was performed in the T-cell medium (complete TexMACS). Briefly, target cell lines were thawed and washed in complete DMEM U2OS (passage 6) red fluorescent protein (RFP)+(e.g., 10 ml DMEM, acridine orange (AO) and propidium iodide (PI) (AOPI) count: 0.276×10⁶/ml viability 93.9%) and A375 RFP+(e.g., 10 ml DMEM, AOPI count: 0.262×10⁶/ml viability 94.9%). Cells were seeded into T75 flasks in a total volume of 20 ml DMEM supplemented with FBS. Effector cells were thawed and washed in complete TexMacs. P-TCR1, P-TCR2, and P-TCR3 T cells were AOPI counted and adjusted to 0.5×10⁶/ml in complete TexMacs. Both target cell lines, e.g., U2OS and A375, were adjusted to 50.000 cells/ml and 100 ul (5000 cells) were plated in IncuCyte Imagelock plates. 50.000 Effector cells were added in 100 ul for a total of 200 ml assay volume.

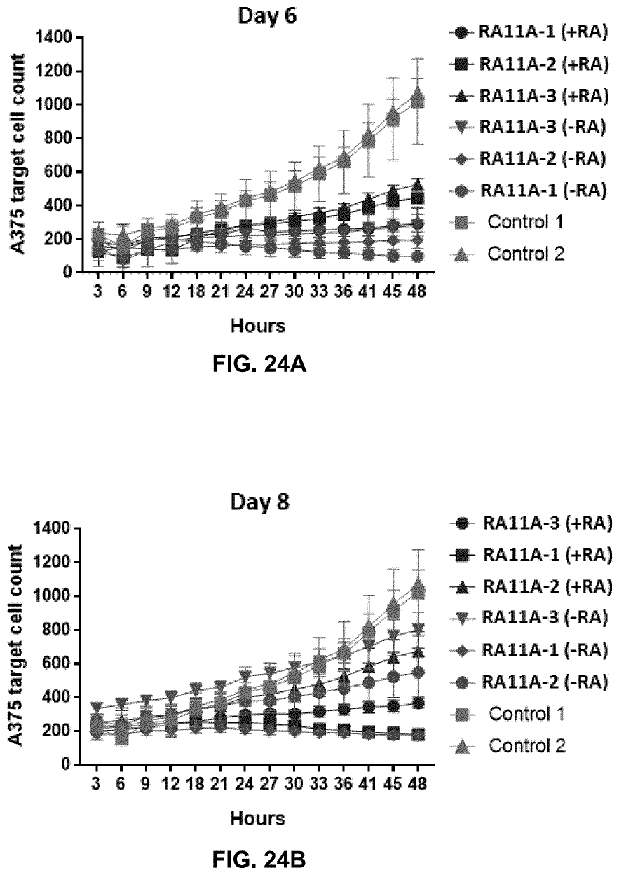

FIG. 24A shows the killing of A375 cells by R11A-1, R11A-2, and R11A-3 T cells with RA (+RA) or without RA (−RA) treatment harvested on Day 6.

FIG. 24B shows the killing of A375 cells by R11A-1, R11A-2, and R11A-3 T cells with RA (+RA) or without RA (−RA) treatment harvested on Day 8.

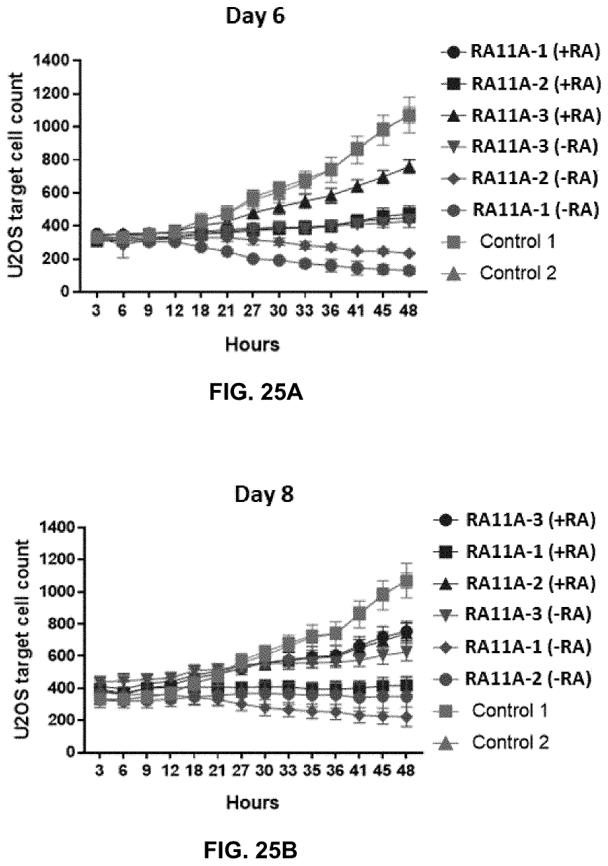

FIG. 25A shows the killing of U2OS cells by R11A-1, R11A-2, and R11A-3 T cells with RA (+RA) or without RA (−RA) treatment harvested on Day 6.

FIG. 25B shows the killing of U2OS cells by R11A-1, R11A-2, and R11A-3 T cells with RA (+RA) or without RA (−RA) treatment harvested on Day 8.

Table 3 summarizes the killing activity of R11A-1, R11A-2, and R11A-3 T cells with RA (+) or without RA (−) as measured by % reduction of target cells at 48 hours in the assay. The controls (e.g., target cells in the absence of the TCR transduced T cells) were set as 100%.

TABLE 3

| | | A375 cells | | U2OS cells | |
|---|---|---|---|---|---|
| | RA | Day 6 | Day 8 | Day 6 | Day 8 |
| R11A-1 T | + | 72% | 64.9% | 58.1% | 29.4% |
| cells | − | 72.9% | 23.6% | 59.8% | 41.5% |
| R11A-2 T | + | 57.4% | 82.7% | 55.8% | 61% |
| cells | − | 81.4% | 83.0% | 78.1% | 79.2% |
| R11A-3 T | + | 49.5% | 35.6% | 29.1% | 30.6% |
| cells | − | 90.6% | 47.5% | 87.9% | 67.5% |

The killing results indicate both target cell lines, e.g., A375 and U2OS cells, were recognized in the killing assay by Day 6 and Day 8 transduced T cells. Against the lower target expressing A375 cell line, Day 6 cells seem more potent than Day 8 cells. This was not observed for the higher target expressing U2OS cell line. The non-RA treated transduced T cells express higher levels of the R11A TCR than RA treated transduced T cells. The RA treated cells are able to kill both high- and low-target expressing cells.

What is claimed:

1. A method of preparing a T cell population comprising
obtaining a population of peripheral blood mononuclear cells (PBMC);
selecting T-cells in the population of the PBMC population;
activating the T-cells with an anti-CD3 antibody and an anti-CD28 antibody
transducing the activated T cells with a viral vector,
expanding the transduced T cells, and
obtaining the expanded T cells,
wherein the expanded T cell are capable of specifically binding a peptide consisting of the amino acid sequence of SLLMWITQC (SEQ. ID. NO. 131) or GVYDGREHTV (SEQ ID NO: 89).

2. The method of claim 1, wherein the activation comprises incubation with Interleukin-2 (IL-2).

3. The method of claim 1, further comprising resting the selected T-cells, optionally in the presence of at least one cytokine.

4. The method of claim 3, wherein the at least one cytokine is interleukin 2 (IL-2), interleukin 7 (IL-7), interleukin 15 (IL-15), interleukin 21 (IL-21), or a combination thereof.

5. The method of claim 3, wherein the at least one cytokine is in an amount between about 1 ng/mL and 500 ng/mL.

6. The method of claim 1, wherein the viral vector is a lentivirus vector.

7. The method of claim 1, wherein the T cells are expanded for 1-15 days.

8. The method of claim 1, wherein the T cells are expanded in the presence of IL-2, IL-7, IL-12, IL-15, or a combination thereof.

9. The method of claim 1, wherein the T cells are CD4+.

10. The method of claim 1, wherein the T cells are CD8+.

11. The method of claim 1, wherein the method further comprises transfecting the T-cells with a nucleic acid encoding CD8.

12. A method of treating a cancer that presents a peptide consisting of the amino acid sequence of SLLMWITQC (SEQ ID NO: 131) or GVYDGREHTV (SEQ ID NO: 89) in a complex with an MHC molecule on the surface of cancer cells, comprising
selecting a patient suffering from the cancer; and
administering a composition comprising the T cell of claim 1,
wherein the cancer is melanoma, ovarian cancer, esophageal cancer, gastric cancer, non-small cell lung cancer (NSCLC), head and neck cancer, or a combination thereof.

13. The method of claim 12, wherein the T cells are autologous to the patient.

14. The method of claim 12, wherein the T cells are allogenic to the patient.

15. The method of claim 12, wherein the dosage of T-cells is about 1×10⁹ to about 1×10¹¹ T cells.

16. The method of claim 12, wherein the composition comprising T-cells is administered by intravenous infusion.

17. The method of claim 1, wherein the expanded T cells are capable of specifically binding a peptide consisting of the amino acid sequence of SEQ ID NO: 131 (SLLMWITQC).

18. The method of claim 17, wherein the viral vector comprises a nucleic acid encoding a T cell receptor (TCR) that binds a peptide consisting of the amino acid sequence of SEQ ID NO: 131 (SLLMWITQC).

19. The method of claim 1, wherein the expanded T cells are capable of specifically binding a peptide consisting of the amino acid sequence of SEQ ID NO: 89 (GVYDGREHTV).

20. The method of claim 19, wherein the viral vector comprises a nucleic acid encoding a TCR that binds a peptide consisting of the amino acid sequence of SEQ ID NO: 89 (GVYDGREHTV).


SEQUENCE LISTING (157 sequences)

SEQ ID NO: 1 - protein, 10 aa, Homo sapiens
YLYDSETKNA

SEQ ID NO: 2 - protein, 9 aa, Homo sapiens
HLMDQPLSV

SEQ ID NO: 3 - protein, 9 aa, Homo sapiens
GLLKKINSV

SEQ ID NO: 4 - protein, 9 aa, Homo sapiens
FLVDGSSAL

SEQ ID NO: 5 - protein, 10 aa, Homo sapiens
FLFDGSANLV

SEQ ID NO: 6 - protein, 9 aa, Homo sapiens
FLYKIIDEL

SEQ ID NO: 7 - protein, 11 aa, Homo sapiens
FILDSAETTTL

SEQ ID NO: 8 - protein, 9 aa, Homo sapiens
SVDVSPPKV

SEQ ID NO: 9 - protein, 8 aa, Homo sapiens
VADKIHSV

SEQ ID NO: 10 - protein, 9 aa, Homo sapiens
IVDDLTINL

SEQ ID NO: 11 - protein, 9 aa, Homo sapiens
GLLEELVTV

SEQ ID NO: 12 - protein, 10 aa, Homo sapiens
TLDGAAVNQV

SEQ ID NO: 13 - protein, 10 aa, Homo sapiens
SVLEKEIYSI

SEQ ID NO: 14 - protein, 9 aa, Homo sapiens
LLDPKTIFL

SEQ ID NO: 15 - protein, 9 aa, Homo sapiens
YTFSGDVQL

SEQ ID NO: 16 - protein, 9 aa, Homo sapiens
YLMDDFSSL

SEQ ID NO: 17 - protein, 9 aa, Homo sapiens
KVWSDVTPL

SEQ ID NO: 18 - protein, 11 aa, Homo sapiens
LLWGHPRVALA

SEQ ID NO: 19 - protein, 11 aa, Homo sapiens
KIWEELSVLEV

SEQ ID NO: 20 - protein, 9 aa, Homo sapiens
LLIPFTIFM

SEQ ID NO: 21 - protein, 9 aa, Homo sapiens
FLIENLLAA

SEQ ID NO: 22 - protein, 11 aa, Homo sapiens
LLWGHPRVALA

SEQ ID NO: 23 - protein, 9 aa, Homo sapiens
FLLEREQLL

SEQ ID NO: 24 - protein, 9 aa, Homo sapiens
SLAETIFIV

SEQ ID NO: 25 - protein, 9 aa, Homo sapiens
TLLEGISRA

SEQ ID NO: 26 - protein, 9 aa, Homo sapiens
ILQDGQFLV

SEQ ID NO: 27 - protein, 10 aa, Homo sapiens
VIFEGEPMYL

SEQ ID NO: 28 - protein, 9 aa, Homo sapiens
SLFESLEYL

SEQ ID NO: 29 - protein, 9 aa, Homo sapiens
SLLNQPKAV

SEQ ID NO: 30 - protein, 9 aa, Homo sapiens
GLAEFQENV

SEQ ID NO: 31 - protein, 9 aa, Homo sapiens
KLLAVIHEL

SEQ ID NO: 32 - protein, 9 aa, Homo sapiens
TLHDQVHLL

SEQ ID NO: 33 - protein, 11 aa, Homo sapiens
TLYNPERTITV

SEQ ID NO: 34 - protein, 9 aa, Homo sapiens
KLQEKIQEL

SEQ ID NO: 35 - protein, 10 aa, Homo sapiens
SVLEKEIYSI

SEQ ID NO: 36 - protein, 11 aa, Homo sapiens
RVIDDSLVVGV

SEQ ID NO: 37 - protein, 9 aa, Homo sapiens
VLFGELPAL

SEQ ID NO: 38 - protein, 9 aa, Homo sapiens
GLVDIMVHL

SEQ ID NO: 39 - protein, 9 aa, Homo sapiens
FLNAIETAL

SEQ ID NO: 40 - protein, 9 aa, Homo sapiens
ALLQALMEL

SEQ ID NO: 41 - protein, 9 aa, Homo sapiens
ALSSSQAEV

SEQ ID NO: 42 - protein, 11 aa, Homo sapiens
SLITGQDLLSV

SEQ ID NO: 43 - protein, 9 aa, Homo sapiens
QLIEKNWLL

SEQ ID NO: 44 - protein, 9 aa, Homo sapiens
LLDPKTIFL

SEQ ID NO: 45 - protein, 9 aa, Homo sapiens
RLHDENILL

SEQ ID NO: 46 - protein, 9 aa, Homo sapiens
YTFSGDVQL

SEQ ID NO: 47 - protein, 9 aa, Homo sapiens
GLPSATTTV

SEQ ID NO: 48 - protein, 11 aa, Homo sapiens
GLLPSAESIKL

SEQ ID NO: 49 - protein, 9 aa, Homo sapiens
KTASINQNV

SEQ ID NO: 50 - protein, 9 aa, Homo sapiens
SLLQHLIGL

SEQ ID NO: 51 - protein, 9 aa, Homo sapiens
YLMDDFSSL

SEQ ID NO: 52 - protein, 9 aa, Homo sapiens
LMYPYIYHV

SEQ ID NO: 53 - protein, 9 aa, Homo sapiens
KVWSDVTPL

SEQ ID NO: 54 - protein, 11 aa, Homo sapiens
LLWGHPRVALA

SEQ ID NO: 55 - protein, 9 aa, Homo sapiens
VLDGKVAVV

SEQ ID NO: 56 - protein, 9 aa, Homo sapiens
GLLGKVTSV

SEQ ID NO: 57 - protein, 9 aa, Homo sapiens
KMISAIPTL

SEQ ID NO: 58 - protein, 11 aa, Homo sapiens
GLLETTGLLAT

SEQ ID NO: 59 - protein, 9 aa, Homo sapiens
TLNTLDINL

SEQ ID NO: 60 - protein, 9 aa, Homo sapiens
VIIKGLEEI

SEQ ID NO: 61 - protein, 9 aa, Homo sapiens
YLEDGFAYV

SEQ ID NO: 62 - protein, 11 aa, Homo sapiens
KIWEELSVLEV

SEQ ID NO: 63 - protein, 9 aa, Homo sapiens
LLIPFTIFM

SEQ ID NO: 64 - protein, 10 aa, Homo sapiens
ISLDEVAVSL

SEQ ID NO: 65 - protein, 10 aa, Homo sapiens
KISDFGLATV

SEQ ID NO: 66 - protein, 11 aa, Homo sapiens
KLIGNIHGNEV

SEQ ID NO: 67 - protein, 9 aa, Homo sapiens
ILLSVLHQL

SEQ ID NO: 68 - protein, 9 aa, Homo sapiens
LDSEALLTL

SEQ ID NO: 69 - protein, 13 aa, Homo sapiens
VLQENSSDYQSNL

SEQ ID NO: 70 - protein, 11 aa, Homo sapiens
HLLGEGAFAQV

SEQ ID NO: 71 - protein, 9 aa, Homo sapiens
SLVENIHVL

SEQ ID NO: 72 - protein, 9 aa, Homo sapiens
YTFSGDVQL

SEQ ID NO: 73 - protein, 9 aa, Homo sapiens
SLSEKSPEV

SEQ ID NO: 74 - protein, 10 aa, Homo sapiens
AMFPDTIPRV

SEQ ID NO: 75 - protein, 9 aa, Homo sapiens
FLIENLLAA

SEQ ID NO: 76 - protein, 9 aa, Homo sapiens
FTAEFLEKV

SEQ ID NO: 77 - protein, 9 aa, Homo sapiens
ALYGNVQQV

SEQ ID NO: 78 - protein, 9 aa, Homo sapiens
LFQSRIAGV

SEQ ID NO: 79 - protein, 11 aa, Homo sapiens
ILAEEPIYIRV

SEQ ID NO: 80 - protein, 9 aa, Homo sapiens
FLLEREQLL

SEQ ID NO: 81 - protein, 10 aa, Homo sapiens
LLLPLELSLA

SEQ ID NO: 82 - protein, 9 aa, Homo sapiens
SLAETIFIV

SEQ ID NO: 83 - protein, 11 aa, Homo sapiens
AILNVDEKNQV

SEQ ID NO: 84 - protein, 9 aa, Homo sapiens
RLFEEVLGV

SEQ ID NO: 85 - protein, 9 aa, Homo sapiens
YLDEVAFML

SEQ ID NO: 86 - protein, 11 aa, Homo sapiens
KLIDEDEPLFL

SEQ ID NO: 87 - protein, 9 aa, Homo sapiens
KLFEKSTGL

SEQ ID NO: 88 - protein, 11 aa, Homo sapiens
SLLEVNEASSV

SEQ ID NO: 89 - protein, 10 aa, Homo sapiens
GVYDGREHTV

SEQ ID NO: 90 - protein, 10 aa, Homo sapiens
GLYPVTLVGV

SEQ ID NO: 91 - protein, 9 aa, Homo sapiens
ALLSSVAEA

SEQ ID NO: 92 - protein, 9 aa, Homo sapiens
TLLEGISRA

SEQ ID NO: 93 - protein, 9 aa, Homo sapiens
SLIEESEEL

SEQ ID NO: 94 - protein, 9 aa, Homo sapiens
ALYVQAPTV

SEQ ID NO: 95 - protein, 10 aa, Homo sapiens
KLIYKDLVSV

SEQ ID NO: 96 - protein, 9 aa, Homo sapiens
ILQDGQFLV

SEQ ID NO: 97 - protein, 9 aa, Homo sapiens
SLLDYEVSI

SEQ ID NO: 98 - protein, 9 aa, Homo sapiens
LLGDSSFFL

SEQ ID NO: 99 - protein, 10 aa, Homo sapiens
VIFEGEPMYL

SEQ ID NO: 100 - protein, 9 aa, Homo sapiens
ALSYILPYL

SEQ ID NO: 101 - protein, 9 aa, Homo sapiens
FLFVDPELV

SEQ ID NO: 102 - protein, 11 aa, Homo sapiens
SEWGSPHAAVP

SEQ ID NO: 103 - protein, 9 aa, Homo sapiens
ALSELERVL

SEQ ID NO: 104 - protein, 9 aa, Homo sapiens
SLFESLEYL

SEQ ID NO: 105 - protein, 9 aa, Homo sapiens
KVLEYVIKV

SEQ ID NO: 106 - protein, 10 aa, Homo sapiens
VLLNEILEQV

SEQ ID NO: 107 - protein, 9 aa, Homo sapiens
SLLNQPKAV

SEQ ID NO: 108 - protein, 9 aa, Homo sapiens
KMSELQTYV

SEQ ID NO: 109 - protein, 11 aa, Homo sapiens
ALLEQTGDMSL

SEQ ID NO: 110 - protein, 11 aa, Homo sapiens
VIIKGLEEITV

SEQ ID NO: 111 - protein, 9 aa, Homo sapiens
KQFEGTVEI

SEQ ID NO: 112 - protein, 9 aa, Homo sapiens
KLQEEIPVL

SEQ ID NO: 113 - protein, 9 aa, Homo sapiens
GLAEFQENV

SEQ ID NO: 114 - protein, 9 aa, Homo sapiens
NVAEIVIHI

SEQ ID NO: 115 - protein, 9 aa, Homo sapiens
ALAGIVTNV

SEQ ID NO: 116 - protein, 12 aa, Homo sapiens
NLLIDDKGTIKL

SEQ ID NO: 117 - protein, 10 aa, Homo sapiens
VLMQDSRLYL

SEQ ID NO: 118 - protein, 9 aa, Homo sapiens
KVLEHVVRV

SEQ ID NO: 119 - protein, 9 aa, Homo sapiens
LLWGNLPEI

SEQ ID NO: 120 - protein, 9 aa, Homo sapiens
SLMEKNQSL

SEQ ID NO: 121 - protein, 9 aa, Homo sapiens
KLLAVIHEL

SEQ ID NO: 122 - protein, 10 aa, Homo sapiens
ALGDKFLLRV

SEQ ID NO: 123 - protein, 11 aa, Homo sapiens
FLMKNSDLYGA

SEQ ID NO: 124 - protein, 10 aa, Homo sapiens
KLIDHQGLYL

SEQ ID NO: 125 - protein, 12 aa, Homo sapiens
GPGIFPPPPPQP

SEQ ID NO: 126 - protein, 9 aa, Homo sapiens
ALNESLVEC

SEQ ID NO: 127 - protein, 9 aa, Homo sapiens
GLAALAVHL

SEQ ID NO: 128 - protein, 9 aa, Homo sapiens
LLLEAVWHL

SEQ ID NO: 129 - protein, 9 aa, Homo sapiens
SIIEYLPTL

SEQ ID NO: 130 - protein, 9 aa, Homo sapiens
TLHDQVHLL

SEQ ID NO: 131 - protein, 9 aa, Homo sapiens
SLLMWITQC

SEQ ID NO: 132 - protein, 11 aa, Homo sapiens
FLLDKPQDLSI

SEQ ID NO: 133 - protein, 10 aa, Homo sapiens
YLLDMPLWYL

SEQ ID NO: 134 - protein, 9 aa, Homo sapiens
GLLDCPIFL

SEQ ID NO: 135 - protein, 9 aa, Homo sapiens
VLIEYNFSI

SEQ ID NO: 136 - protein, 11 aa, Homo sapiens
TLYNPERTITV

SEQ ID NO: 137 - protein, 9 aa, Homo sapiens
AVPPPPSSV

SEQ ID NO: 138 - protein, 9 aa, Homo sapiens
KLQEELNKV

SEQ ID NO: 139 - protein, 11 aa, Homo sapiens
KLMDPGSLPPL

SEQ ID NO: 140 - protein, 9 aa, Homo sapiens
ALIVSLPYL

SEQ ID NO: 141 - protein, 9 aa, Homo sapiens
FLLDGSANV

SEQ ID NO: 142 - protein, 10 aa, Homo sapiens
ALDPSGNQLI

SEQ ID NO: 143 - protein, 9 aa, Homo sapiens
ILIKHLVKV

SEQ ID NO: 144 - protein, 9 aa, Homo sapiens
VLLDTILQL

SEQ ID NO: 145 - protein, 9 aa, Homo sapiens
HLIAEIHTA

SEQ ID NO: 146 - protein, 9 aa, Homo sapiens
SMNGGVFAV

SEQ ID NO: 147 - protein, 9 aa, Homo sapiens
MLAEKLLQA

SEQ ID NO: 148 - protein, 9 aa, Homo sapiens
YMLDIFHEV

SEQ ID NO: 149 - protein, 11 aa, Homo sapiens
ALWLPTDSATV

SEQ ID NO: 150 - protein, 9 aa, Homo sapiens
GLASRILDA

SEQ ID NO: 151 - protein, 9 aa, Homo sapiens
SYVKVLHHL

SEQ ID NO: 152 - protein, 9 aa, Homo sapiens
VYLPKIPSW

SEQ ID NO: 153 - protein, 9 aa, Homo sapiens
NYEDHFPLL

SEQ ID NO: 154 - protein, 9 aa, Homo sapiens
VYIAELEKI

SEQ ID NO: 155 - protein, 12 aa, Homo sapiens
VHFEDTGKTLLF

SEQ ID NO: 156 - protein, 9 aa, Homo sapiens
VLSPFILTL

SEQ ID NO: 157 - protein, 9 aa, Homo sapiens
HLLEGSVGV